(19) United States
(12) Patent Application Publication
(10) Pub. No.: US 2011/0212501 A1
(43) Pub. Date: Sep. 1, 2011

(54) 3-DIMENSIONAL MULTI-LAYERED HYDROGELS AND METHODS OF MAKING THE SAME

(75) Inventor: Seung-Schik Yoo, Wellesley, MA (US)

(73) Assignee: THE BRIGHAM AND WOMEN'S HOSPITAL, INC., Boston, MA (US)

(21) Appl. No.: 13/063,502

(22) PCT Filed: Sep. 14, 2009
(86) PCT No.: PCT/US2009/056777

§ 371 Date: May 20, 2011

Related U.S. Application Data
(60) Provisional application No. 61/096,437, filed on Sep. 12, 2008.

(51) Int. Cl. C12N 11/00; B05D 1/38; B05D 3/10; B05D 5/00

(52) U.S. Cl. 435/174; 427/337; 427/338; 427/340

(57) ABSTRACT

Embodiments of the invention provide three dimensional multi-layered hydrogel constructs with embedded channels, living cells and bioactive agents, and methods for making three dimensional multi-layered hydrogel constructs. The constructs can have bioactive agents to support the living cells. The multi-layered constructs can have channels for perfusion purposes and layers of different hydrogel materials.

### CROSS REFERENCE TO RELATED APPLICATIONS

This application claims benefit under 35 U. S. C. § 119(e) of the U.S. provisional applications No. 61/096,437 filed on Sep. 12, 2008, the contents of which are incorporated herein by reference in its entirety.

### BACKGROUND OF INVENTION

A significant part of tissue engineering (TE) is concerned with the fabrication of biomaterials as replacement tissues and the development of biomedical devices. The fabricated replacement tissues are engineered to repair congenital defects, diseased tissues, skin wounds and the likes. Replacement tissues are often comprised of biodegradable scaffold engineered with specific desired mechanical properties, are seeded with appropriate cells, and can be supplemented with additional bioactive agents such as growth factors so that, on implantation in vivo, the engineered replacement tissues undergo remodeling and maturation into functional tissue. Examples of replacement tissues include blood vessels, cardiovascular substitutes, bladder, skin, and cartilage.

Despite advances in this field, TE still faces major constraints. Most tissues in the human body are composed of more than one cell type and these cells are embedded within different extracellular matrices. Moreover, these tissues are stratified, with different cell types having specific spatial distribution within the tissue. For example, spatial distribution of annular endothelial cells is required for the development of a functional microvascular network for the efficient delivery of oxygen and nutrients, and the removal of waste materials. Currently, modern TE methods have not been able to engineer replacement tissues that reproduce similar stratifications found in naturally occurring tissue.

While the newer methods of cell ink-jet printing and solid freeform fabrication have allowed the deposition of cells on TE matrices in layer-by-layer fashion, adherence of cells to polymerized matrices remains problematic. The deposited cells and hydrogel precursors are prone to being washed away during the bulk application of the binder or cross-linking agent, hence the desired spatial distribution of cross-linked hydrogels and living cells in the replacement TE tissues cannot be realized. When UV, laser or heat is used for initiating the polymerization or cross-linking of the TE matrices, a significant number of cells can be killed or altered on the account of the UV, laser-emitted energy or heat. Often, when the cells are not washed away, the cells are not completely embedded within the matrices. The cells are instead found in hollow cavities formed by matrix materials that had polymerized too rapidly. When cells are printed on unpolymerized matrix material and the cross-linking agent is applied in a bulk fashion, the exterior surface of the unpolymerized matrix material that comes in contact with the cross-linking agent first tends to polymerize quickly, while the interior of the unpolymerized matrix material that is not in direct in contact with the cross-linking agent undergoes incomplete polymerization. This results in a shell of polymerized matrix material encasing a core of unpolymerized or partially polymerized matrix material and cells. The shell prevents additional cross-linking agent from penetrating into the core. With repeated processing during the multi-layered fabrication process where there is repeated bulk application of liquid matrix material, the unpolymerized matrix material gets washed away, leaving behind a hollow cavity filled with cells. If the amount of applied cross-linking agent, in an aqueous form, is excessive compared to the amount of unpolymerized matrix material, e. g. hydrogel precursor, mechanical instability occurs in the TE construct. Mechanical instability is a major obstacle to the 3D construction of desired cell-hydrogel composites. Hence, innovative methods of embedding living cells in TE constructs are needed.

### SUMMARY OF THE INVENTION

Embodiments of the invention are based on the discovery that a very fine aerosol mist of a cross-linking agent, when applied to a substrate or the surface of the substrate, can be use to partially polymerized hydrogel precursor material on the same substrate and/or surface. The small amount of cross-linking agent produces a partially polymerized and not a fluidly mobile hydrogel layer. The partial polymerization is sufficient to hold the hydrogel in a specific spatial orientation in which it was printed in freeform fabrication. If desired, living cells can then be printed on to the partially polymerized hydrogel. A second very fine aerosol mist of a cross-linking agent is then applied to complete the polymerization process of the partial polymerized hydrogel, therefore fully encapsulating the cells. In this way, living cells can be strategically and spatially distributed within a single layer of hydrogel material. When combined with computer-assisted design, this method allows the construction of a custom-made, multi-layered cell-hydrogel TE construct according to the required shape and size of a replacement tissue.

Accordingly, the invention provides for a method of making a three dimensional multi-layered hydrogel construct, the method comprising the steps of: (a) applying a first nebulized layer of cross-linking agent on a substrate; (b) depositing a layer of hydrogel precursor on top of the first nebulized layer of cross-linking material, wherein the hydrogel precursor cross-links upon contact with the nebulized layer of cross-linking material to form a partially cross-linked gel; (c) applying a second nebulized layer of cross-linking agent on top of the partially cross-linked gel of step (b), thereby promoting completing cross-linking of the layer of hydrogel of step (b); and (d) repeating alternating step b followed by step (c).

In one embodiment, the hydrogel layer is deposited via a drop-by-drop on-demand printing or continuous extrusion of the precursors.

In one embodiment, the nebulized cross-linking material comprises 1-100 micrometer sized droplets. The size can range from 1-100, including all the whole integers and fractions thereof.

In one embodiment, the repeating alternating step (b) followed by step (c) is repeated 1-20 times, including all the whole integers between the number 1 and 20. In other embodiments, the repeating alternating step (b) followed by step (c) is repeated at least 5 times, at least 6, at least 7, at least 8, at least 9, at least 10, at least 11, at least 12, at least 13, at least 14 at least 15, at least 16, at least 17, at least 18, at least 19, or at least 20 times.

In one embodiment, the multi-layered three dimensional construct comprises more than one type of hydrogel. The hydrogel precursor includes, for example, collagen, gelatin, fibrinogen, chitosan, hyaluronan acid, alginate, poly-ethylene glycol, lactic acid, and N-isopropyl acrylamide. Different cross-linking/gelation agents are used for the respective hydrogel. In some embodiments, the multi-layer construct has alternating different types of hydrogel materials, for example, one layer of collagen followed by one layer of fibrin (gel of fibrinogen, thrombin and heparin), followed by a second layer of collagen. In one embodiment, the multi-layered three dimensional construct is a composite comprising collagen layers and fibrin layers.

In one embodiment, the method further comprises depositing living cells on a layer of hydrogel precursor after step (b) but prior to step (c). In some embodiments, more than one cell type is deposited in the multi-layered three dimensional construct. Cell types useful in the invention include, but not limited to, for example, stems cells, pancreatic progenitor cells, neuronal cells, vascular endothelial cells, hair follicular stem cells, mesenchymal cells, and smooth muscle cells.

In one embodiment, the substrate for making of the multi-layered 3D TE construct is flat. In another embodiment, the substrate is contoured.

In another embodiment, the substrate for making of the multi-layered 3D TE is biological. In yet another embodiment, the substrate for making of the multi-layered 3D TE is non-biological.

As used herein, the term “non-biological” refers to a substrate that is comprised solely of synthetic materials. “Non-biological” also refers to not involving, relating to, or derived from biology or living organisms.

As used herein, the term “biological refers to a substrate that is comprised of materials that involves, relate to, or are derived from biology or living organisms. For example, extracellular matrices made naturally by living cells, a layer of cells cultured on a culture dish or on a TE scaffold, or a living tissue, organ or body part.

In some embodiments, the three dimensional multi-layered hydrogel construct further comprise channels. In certain embodiments, the channels can be perfused with fluids such as culture media, plasma, artificial blood or blood to nourish the construct in culture.

In one embodiment, provided herein is a three dimensional multi-layered hydrogel construct that comprises at least 10 layers of hydrogel material and at least one type of living cells. In some embodiments, there can be more than one type of cells on a single layer of hydrogel material. In other embodiments, the cells can be deposited on different layers of hydrogel material. The construct can include one type of hydrogel material or multiple types of hydrogel material.

In one embodiment, the three dimensional multi-layered hydrogel construct includes fibroblast and keratinocytes. In a further embodiment, hair follicular stem cells are incorporated into the construct. In other embodiments, the construct comprise neurons, aastrocytes, and/or neural stem cells.

In one embodiment, the three dimensional multi-layered hydrogel construct includes cells types that are vascular endothelial progenitor cells and smooth muscle progenitor cells or mesenchymal stem cells.

In one embodiment, the three dimensional multi-layered hydrogel construct includes pancreatic endothelial progenitor cells and mesenchymal stem cells.

In further embodiments, the three dimensional multi-layered hydrogel construct comprises bioactive agents such as, for example, growth factors, differentiation factors and/or cytokines. In yet further embodiments, the three dimensional multi-layered hydrogel constructs comprise therapeutic agents.

### BRIEF DESCRIPTION OF THE DRAWINGS

FIG. 1 shows the schematic of implementation of the 3D tissue printer. Input images can be chosen from variety of sources including CAD files or 3D radiological images. In-house software generated dispenses coordinates/vectors as well as the printing sequence whereby the user controlled the dispensing resolutions and gradients through graphic-user-interface (GUI). The printing information, after conversion to the robot controller language, is fed to the printer. The volume of droplet was adjusted independently by controlling the pneumatic pressure to the fluid paths or the opening duration for the microvalve.

FIG. 2 shows the schematic for the making of a multi-layered composition of hydrogels and cells using administration of the nebulized cross-linking agent on the printed hydrogel precursors. Robotic stages control the timing and location of the cell/hydrogel droplets.

FIG. 3 shows the schematic of layer-by-layer printing of the multi-layered skin cells and collagen (left panel) including its side view (right panel). Human fibroblasts (hFBs) were printed in the 2nd collagen layer, and six layers of collagen were printed over the FBs. Human keratinocytes (hKCs) were printed in the 8th layer of collagen and two layers of collagen were used to cover the KC layer.

FIG. 4A shows the negative mold with 3D contour for a PDMS mold of 3D skin wound model. The aluminum cast was prepared to imprint this negative mold and used to construct PDMS mold.

FIG. 4B shows a prepared PDMS mold of 3D skin wound model. Multi-layers of collagen and skin cells were printed onto the 3D mold surface of the wound model.

FIG. 4C shows the used image sequence for printing of collagen and cells.

FIGS. 5A-F shows images of a multi-layered hydrogel printed with living cells.

FIGS. 5A-C shows the culture images at Day 1 of hFBs printed in 300 μm, 400 μm, and 500 μm resolution.

FIGS. 5D-F shows the culture images at Day 8 of hFBs printed in 300 μm, 400 μm, and 500 μm resolution. Inter-dispensing distance of 300 μm showed confluent cell density on Day 8.

FIG. 5G shows the on-demand 2D printing of a plus shape, with dotted lines indicating the printing profile.

FIG. 6 shows cell images after multi-layered printing of hFB and hKC on a tissue culture dish. (A) Volume rendered immunofluorescent images of multi-layered printing of hKC and hFB and its projection of (B) keratin-containing KC layer and (C)β-tubulin-containing hKC and hFB. The inter layer distance of approximately 75 μm was observed. Bright field images on (D) hKC layer and (E) hFB layer also confirmed the immunohistochemistry (IHC) findings.

FIG. 7A shows an image (obtained in Day 1 of culture) after the multi-layered printing of hFB and hKC on PDMS mold of 3D skin wound model.

FIG. 7B shows the stereomicroscopic top view of printed multi-layer cell-collagen composite on the PDMS mold of 3D skin wound model.

FIG. 7C shows the bright-field images of hKC in the printed multi-layer cell-collagen composite on the PDMS mold of 3D skin wound model.

FIG. 7D shows the bright-field images of the hFB layer of the printed multi-layer cell-collagen composite on the PDMS mold of 3D skin wound model.

FIG. 8 shows the schematic procedure of constructing a 3D hydrogel block containing micro-fluidic channels (herein defined as ‘fluidic hydrogel structure’) using the 3D cell-hydrogel printer.

FIG. 9A shows the mean droplet volume with standard error of distilled water (N=5), fibroblast cell suspension (N=5), 2 mg/mL (0.5×) and 1.33 mg/mL (0.3×) of collagen precursor(N=5) with increase of pneumatic pressure to microvalve when the valve opening duration is 450 μs.

FIG. 9B shows the mean droplet volume with standard error of distilled water (N=5), fibroblast cell suspension (N=5), 2 mg/mL (0.5×) and 1.33 mg/mL (0.3×) of collagen precursor(N=5) with increase of pneumatic pressure to microvalve when the valve opening duration is 600 μs

FIG. 9C shows the mean droplet volume with standard error of distilled water (N=5), fibroblast cell suspension (N=5), 2 mg/mL (0.5×) and 1.33 mg/mL (0.3×) of collagen precursor(N=5) with increase of pneumatic pressure to microvalve when the valve opening duration is 750 μs.

FIG. 9D shows the mean droplet volume of 7 wt % gelatin (at 40° C.) (N=8) measured with pressure range from 6 psi to 13 psi using different valve opening duration of 450 μs, 600 μs and 750 μs.

FIG. 10 shows the image of gelated gelatin channel (between the dotted lines) in collagen groove under bright field microscope. At current 3D cell-hydrogel printing set-up, the nominal printable line width of gelatin channel was around 400 μm (The scale bar=200 μm).

FIG. 11A shows the images of the printed line patterns of gelatin channels on tissue culture dish with 4 psi pressure, 400 μs valve opening time, and 500 μm printing resolution(scale bar=250 μm).

FIG. 11B shows the images of the printed line patterns of gelatin channels on tissue culture dish with 4 psi pressure, 400 μs valve opening time, and 400 μm printing resolution (scale bar=250 μm).

FIG. 11C shows the images of the printed line patterns of gelatin channels on tissue culture dish with 4 psi pressure, 400 μs valve opening time, and 300 μm printing resolution (scale bar=250 μm).

FIG. 11D shows the printed gelatin line pattern (between the dotted lines) in the collagen groove.

FIG. 11E shows air bubbles injected into the gelatin channel for inspection after selective gelatin removal under stereomicroscopy. The printed gelatin line pattern was embedded in multi-layered collagen scaffold and selectively removed.

FIG. 12A shows a “plus” pattern of gelatin channel constructed in 2nd layer of three multi-layered collagen scaffold fluidic hydrogel structure having channels using the 3D cell-hydrogel printer and visualized in grey filled channel. The upper picture depicts drawings of the designs and the lower rows are those of real constructions. To visualize the gelatin channels in collagen scaffold, 7 wt % gelatin mixed with colored microbeads is used.

FIG. 12BA shows a rotary pattern of gelatin channel was formed in 2nd layer of three multi-layered collagen scaffold.

FIG. 12C shows a multi-layered rotary-shaped and cross patterns of gelatin channels were built in five multi-layered collagen scaffold.

FIG. 13A-D show the illustrated locations of printed cells used in the viability measurements and plots showing the percentage of cell viability (with respect to the total amounted of printed cells) from twelve areas between channeled hydrogel and the control hydrogel block, measured 36 hours of perfusion.

FIG. 14A-B show the schematics of hFB-laden collagen scaffold construction without (FIG. 14A) and with (FIG. 14B) embedding and removal of printed sacrificial gelatin channel. The figure is not drawn in scale.

FIG. 14C-D show the FB viability inspected locations (a vertical section at M-M′) in the collagen scaffolds without and with inside media perfusion. Capital letters of (A), (B) and (C) indicate the horizontal distances of <1 mm, 2.5 mm, and 5 mm, respectively from scaffold center. Lower-case letters of (a), (b), and (c) indicate the vertical distances of 400 μm, 200 μm, and 0 μm, respectively from scaffold bottom

FIG. 14E-F show the measured FB viability with standard error bar (n=12, with respect to the total FB cells) at the inspected locations after 36 hours of culture without and with media perfusion.

FIG. 15A shows the schematic of single-layer patterning of neuronal cells as a ‘ring’ pattern of neurons with a 3 mm diameter in a single collagen layer.

FIG. 15B shows the schematic of single-layer patterning of neuronal cells in a ‘cross’ pattern of neurons of 6 mm long in a single collagen layer.

FIG. 15C shows the schematic of a multilayer ring patterning of neuronal cells for three rings of neurons, wherein one ring of cells are printed in each of the layers.

FIG. 15D shows the schematic of a multilayer ‘cross’ patterning of neuronal cells and astrocytes.

FIG. 16A shows fluorescent image of printed neurons in a single layer of collagen scaffold at printing resolutions of 150 μm taken after day 15 in culture.

FIG. 16B shows fluorescent image of printed neurons in single layer of collagen scaffold at printing resolutions of 250 μm taken after day 15 in culture.

FIG. 16C shows bright field images of printed astrocytes in single layer of collagen scaffold with printing resolutions of 400 μm after day 3 in culture.

FIG. 16D shows bright field images of printed astrocytes in single layer of collagen scaffold with printing resolutions of 600 μm after day 3 in culture.

FIG. 17A shows the fluorescent live-staining images of cultured neurons in a single-layered collagen scaffold on day 15 after printing in a printed ‘ring’ pattern of neurons with 3 mm diameter in a single layer of collagen scaffold (Inset: A part of the printed ‘ring’).

FIG. 17B shows the fluorescent live-staining images of cultured neurons in a single-layered collagen scaffold on day 15 after printing in a printed ‘cross’ pattern of neurons 6 mm long

FIG. 17C shows a vertically-projected image of printed cell block in collagen through 3D volume.

FIG. 17D shows a multilayer patterning of three neuron rings within eight layers of collagen.

FIG. 17E shows a magnified side view of distinct layers of printed rings of neurons in FIG. 17D.

FIG. 18A shows an immunostaining image of 3D multilayered patterning of the cells in six layers of collagen imaged in day 7. The vertical line composed of astrocytes located in the first collagen layer and horizontal line composed of neurons embedded in the fifth collagen layer (counted from bottom).

FIG. 18B shows an immunostaining image of co-cultured neurons and astrocytes, wherein printed neurons and astrocytes are both in a single-layer collagen scaffold after day 12 of culture.

### DETAILED DESCRIPTION OF THE INVENTION

Embodiments of the invention are based on the discovery that a very fine aerosol mist of a cross-linking agent can be use to partially polymerized hydrogel precursor material on a substrate. The small amount of cross-linking agent produces only a partially polymerized and not fluidly mobile hydrogel layer. The partial polymerization is sufficient to hold the hydrogel in a specific spatial orientation in which it was printed in freeform fabrication.

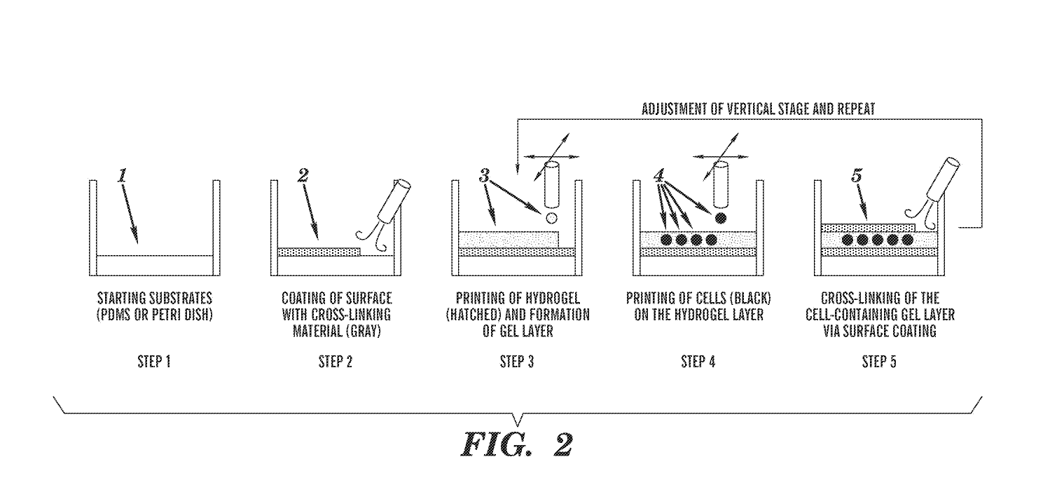

In one embodiment, as illustrated in FIG. 2, the very fine aerosol mist of a cross-linking agent [2] is first applied on to the surface of a substrate [1] by nebulization using, for example, an ultrasonic transducer (14 mm in diameter operating at 2.5 MHz resonance frequency) (see FIG. 2, step 2). Next, droplets of unpolymerized hydrogel precursor material [3] are applied on this nebulized layer of cross-linking agent (FIG. 2, step 3). The droplets of the hydrogel precursor are positioned in a definite spatial arrangement corresponding to the desired shape of the tissue engineered construct or device being made. The amount of cross-linking agent is sufficient to initiate the polymerization process of the hydrogel precursor immediately when the cross-linking agent and hydrogel precursor come in contact with each other. This allow freeform fabrication by ink-jet printing and patterning of 3-D hydrogel-based TE devices or construct to be achieved without the need for an exterior mold.

Normally, when two printed droplets of hydrogel precursor are very close together (˜100 μm) or even overlap each other, the fluid droplets tend to merge into one bigger droplet if the droplets are not immobilized immediately in space, for example, by cross-linking. This phenomenon makes it difficult to pattern a 3-D hydrogel TE construct of a specific shape. The nebulized layer of cross-linking agent serves to prevent this merging of two droplets during freeform fabrication. At the same time, the carefully controlled amount of cross-linking agent does not result in such a rapid polymerization as seen in the bulk application methods that are currently being used, thus circumventing the problems associated with bulk application of cross-linking agents. In bulk application, the printed hydrogel precursor is dipped or submerged into a container holding the cross-linking agent. The bulk application method is largely the cause of printed cells being washed away and/or displacement, of printed cells not being fully embedded within polymerized matrices, and misshaped 3D TE construct.

Since, the small amount of cross-linking agent produces only a partially polymerized, but not fluidly mobile hydrogel layer, cells [4] can be strategically printed on this partially polymerized hydrogel layer (FIG. 2, step 4). When a second aerosol mist of the cross-linking agent [5] is applied over the first partially polymerized hydrogel layer with strategically printed living cells (FIG. 2, step 5), the second layer of cross-linking agent promotes the complete polymerization of the first hydrogel layer, thereby embedding and encapsulating the living cells in that layer. Here, there is very limited opportunity for accidentally washing away or displacement of the cells on the hydrogel layer.

When a second layer of hydrogel precursor is printed on the second nebulized layer of cross-linking agent, the second nebulized layer of cross-linking agent promotes the partial cross-linking of the second hydrogel precursor layer. The repeated and alternating application of a nebulized layer of cross-linking agent followed with a layer of hydrogel precursor allows the inventor to create a multi-layer 3 D TE construct of at least ten layers or even more than ten layers. Depending on the hydrogel material used and the thickness of the hydrogel layers, the multi-layer 3 D TE construct can have as many as 20 layers and possibly more. Additionally, different hydrogel precursor material can be used for different layers and the cross-linking agent can be changed accordingly.

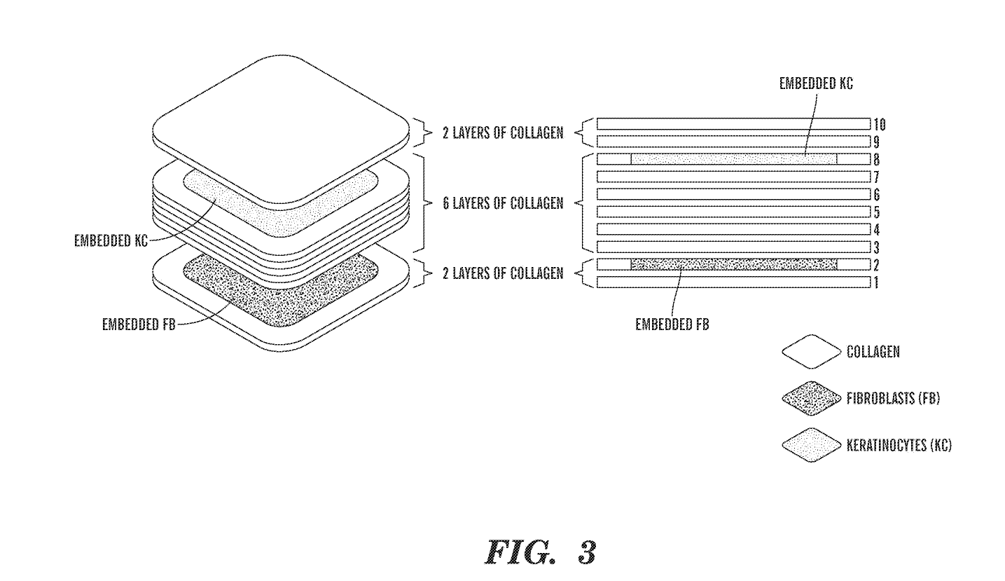

Cells can be strategically printed on all, some or none of the hydrogel layers. Different types of cells can be printed within a single TE construct, embedded in different hydrogel layers, and can also be differentially and spatially distributed in the different hydrogel layers. In addition, more than one type of cells can be printed within single layer of hydrogel. In FIG. 3, the inventor shows a TE construct having ten layers of collagen, wherein fibroblasts are printed in layer 2, keratinocytes are printed in layer 10, and the collagen layers 3-9 do not have cells.

The process of repeated and alternating application of a nebulized layer of cross-linking agent followed with a layer of hydrogel precursor can be integrated with a computer aided design program that controls the multi-inkjet printing nozzles for dispensing different hydrogel precursors and cell types. In a typical 3 D printing apparatus, the dispensing nozzles are used in conjunction with a computer-controlled stage that is movable in the X-, Y- and Z-axes, to produce a multi-layer three dimensional tissue engineering construct of a specified dimension and shape. Computer-assisted-freeform fabrication methods are known to one of ordinary skill in the art, is described herein and are also found in U.S. Pat. application Nos. US 2006/0105011 and US 2006/0160250. One skilled in the art can easily modify the conventional computer-assisted-freeform fabrication methods to integrate a nebulization of cross-linking agent steps into the program.

Accordingly, embodiments of the invention provide methods to create multi-layered tissue engineered (TE) composites that mimic that of natural tissues. Natural tissues of an organism comprise many different cell types, matrix materials (connective tissues) and have various spatial distributions of different cell types and matrix matrices. For example, the skin is a stratified tissue consisting of the epidermis, dermis, and hypodermis layers, with each layer further subdivided into layers having various cells and cell matrices etc., e. g. fibroblasts (FB) and keratinocytes (KC).

The inventor developed and implemented a novel 3D cell-hydrogel printer for on-demand 3D multi-layered cell-hydrogel printing to create, as a model for proof of principle, a stratified skin model that can be used for skin regeneration and wound-specific tissue engineered skin products. The 3D cell-hydrogel printer uses electromechanical microvalve that results in high cell viability in the printed human FB and KC cells embedded within the collagen hydrogel layers, where collagen is the scaffold material.

The inventors also demonstrate that the method is applicable to a neural tissue model wherein neurons, neural stem cells and astrocytes are printed/co-cultured in a single layer of hydrogel or on different layers of hydrogel in the 3D multi-layered construct (See Example 8).

In example 9, the inventors demonstrate a model 3D multi-layered construct cell-hydrogel with alternating collagen layers and fibrin layers. The collagen layer is printed with neural stem cells (NSCs) and the fibrin contains bioactive agent VEGF. The NSCs in the collagen layer response to the VEGF in the fibrin layer.

In one embodiment, the invention provides a method of making a three dimensional (3D) multi-layered hydrogel construct, the method comprises the steps of: (a) applying a first nebulized layer of cross-linking agent on a substrate; (b) depositing a layer of hydrogel precursors on top of at least a portion of the first nebulized layer of cross-linking agent. The hydrogel precursor cross-links upon contact with the nebulized layer of cross-linking agent to form a partially cross-linked gel; (c) applying a second nebulized layer of cross-linking agent on top of the partially cross-linked gel of step (b). This promotes completion of cross-linking of the layer of hydrogel of step (b); and (d) repeating alternating step (b) followed by step (c).

The nebulized cross-linking agent comprises droplets of about 1-100 micrometer size in diameter. All whole integers and fractions thereof between numbers 1-100 are also contemplated. The size of the cross-linking droplets is important. It must not be too small (about <1 μm in diameter) or then there will be insufficient amount cross-linking agent to at least partially polymerize the deposited hydrogel precursor and hold the printed pattern during the on-demand printing. At the same time, the droplet should not be too large either (about >100 μm in diameter) for that can lead to rapid polymerization of the printed hydrogel precursor, giving rise to a fully polymerized hydrogel layer as oppose to a partially polymerized hydrogel layer. Larger droplets of cross-linking agent can distort the printed hydrogel precursor pattern due to the merging together of large droplets of cross-linking agent and/or merging together of droplets of cross-linking agent and the printed hydrogel precursor. Therefore, in some embodiments, the ideal droplet size of a nebulized cross-link agent is about 1 to 100 micrometer in diameter.

In one embodiment, the hydrogel precursor is deposited as droplets (FIG. 2, step 3). In another embodiment, the hydrogel precursor is deposited as droplets by a drop-by-drop on-demand printing. In a further embodiment, the hydrogel precursor is deposited as a continuous tube. In some aspects, the deposition of the hydrogel precursor is determined by the operator of the dispensing apparatus described herein and the on-demand feature of the methods described here. In one embodiment, the positions of the droplets or continuous extruded tube of hydrogel precursor that is deposited is controlled by the computer-assisted program, which is specified by the particular construct or composite structure to be made, and the specifications of the construct is entered into the computer program. For example, a square construct of 2×2×0.5 mm is required. The specification is entered into the program by the operator and the dispensing apparatus described herein will dispense droplets or continuous extruded tube of hydrogel precursor to cover a surface area of 2×2 mm over the nebulized layer of cross-linking agent.

In some embodiments, the thickness of the layers of hydrogel in a 3 D multi-layered TE construct is about 5-100 μm for each layer. All whole integers between 5-100 and fractions thereof are also contemplated.

In some embodiments, more than one layer of hydrogel precursor is deposited before the application of the nebulized cross-linking agent on top of the hydrogel precursor to complete the polymerization process. In some embodiments, the number of layers of hydrogel precursor deposited prior to the subsequent nebulizing cross-linking agent is between about one to ten, including all the whole integers between the number one and ten.

In some embodiments, the method of making a 3 D multi-layered hydrogel construct comprises repeating the steps of applying the nebulizing layer of cross-linking agent and overlying the hydrogel precursor on top of the cross-linking agent for at least 5 times, at least 6 times, at least 7 times, at least 8 times, at least 9 times, at least 10 times, at least 11 times, at least 12 times, at least 13 times, at least 14 times, at least 15 times, at least 16 times, at least 17 times, at least 18 times, at least 19 times or at least 20 times. The steps of applying the nebulizing layer of cross-linking agent and overlying the hydrogel precursor on top of the cross-linking agent are performed in an alternating fashion.

In some embodiments, the method of making a 3 D multi-layered hydrogel construct comprises repeating the steps of applying the nebulizing layer of cross-linking agent and overlying the hydrogel precursor on top of the cross-linking agent for at least 10 times.

In some embodiments, the multi-layered 3 D constructs made by the methods described herein comprise more than one type of hydrogel material. Polymerized hydrogel precursor form polymers. Hydrogels have many desirable properties for biomedical applications. For example, they can be made nontoxic and compatible with tissue, and they are usually highly permeable to water, ions and small molecules. Tonically cross-linkable polymers can be anionic or cationic in nature and include but not limited to carboxylic, sulfate, hydroxyl and amine functionalized polymers, normally referred to as hydrogels after being cross-linked. The term “hydrogel” indicates a cross-linked, water insoluble, water containing material.

Suitable cross-linkable polymers or hydrogels which can be used in the present invention include but are not limited to one or a mixture of polymers selected from the group consisting of glycosaminoglycan, silk, fibrin, MATRIGEL®, poly-ethyleneglycol (PEG), polyhydroxy ethyl methacrylate, polyvinyl alcohol, polyacrylamide, poly (N-vinyl pyrolidone), poly glycolic acid (PGA), poly lactic-co-glycolic acid (PLGA), poly e-carpolactone (PCL), polyethylene oxide, poly propylene fumarate (PPF), poly acrylic acid (PAA), hydrolysed polyacrylonitrile, polymethacrylic acid, polyethylene amine, alginic acid, pectinic acid, carboxy methyl cellulose, hyaluronic acid, heparin, heparin sulfate, chitosan, carboxymethyl chitosan, chitin, pullulan, gellan, xanthan, collagen, gelatin, carboxymethyl starch, carboxymethyl dextran, chondroitin sulfate, cationic guar, cationic starch as well as salts and esters thereof. Polymers listed above which are not ionically cross-linkable are used in blends with polymers which are ionically cross-linkable.

In some aspects, some of the preferred hydrogels include one or a mixture of collagen, alginic acid, pectinic acid, carboxymethyl cellulose, hyaluronic acid, chitosan, polyvinyl alcohol and salts and esters thereof. Preferred anionic polymers are alginic or pectinic acid; preferred cationic polymers include chitosan, cationic guar, cationic starch and polyethylene amine. Other preferred polymers include esters of alginic, pectinic or hyaluronic acid and C2 to C4 polyalkylene glycols, e.g. propylene glycol, as well as blends containing 1 to 99 wt % of alginic, pectinic or hyaluronic acid with 99 to 1 wt % polyacrylic acid, polymethacrylic acid or polyvinylalcohol. Preferred blends comprise alginic acid and polyvinylalcohol. Examples of mixtures include but are not limited to a blend of polyvinyl alcohol (PVA) and sodium alginate and propyleneglycol alginate.

The cross-linking ions used to crosslink the polymers can be anions or cations depending on whether the polymer is anionically or cationically cross-linkable. Appropriate cross-linking ions include but not limited to cations selected from the group consisting of calcium, magnesium, barium, strontium, boron, beryllium, aluminum, iron, copper, cobalt, lead and silver ions. Anions can be selected from but not limited to the group consisting of phosphate, citrate, borate, succinate, maleate, adipate and oxalate ions. More broadly, the anions are derived from polybasic organic or inorganic acids. Preferred cross-linking cations are calcium, iron, and barium ions. The most preferred cross-linking cations are calcium and barium ions. The most preferred cross-linking anion is phosphate. Cross-linking can be carried out by contacting the polymers with a nebulized droplet containing dissolved ions. One of ordinary skill in the art will be able to select appropriate cross-linking agent for the respective hydrogel used in the making of a multi-layer TE construct. For example, the gelation of collagen or alginate occurs in the presence of ionic cross-linker or divalent cations such as Ca²⁺, Ba²⁺ and Sr²⁺.

In one embodiment, the hydrogel is fibrin which is made of fibrinogen, thrombin and heparin.

In some embodiments, the hydrogels are modified to improve cell adhesion properties and more closely mimic the tissue structure that the multi-layered 3 D TE construct is being created for. For example, hydrogels can be conjugated with cell-binding motifs such as the peptide sequence Arg—Gly—Asp (RGD) on the precursor. Other ligands from fibronection, vitronection and laminin can also be used. The RGD peptide sequence can be attached to synthetic substrates, scaffold materials, and hydrogel precursors to promote cell attachment (Massia, S. P.; Hubbell, J. A. Cytotechnology, 1992, 10, 189). One ordinary skilled artisan in the art can conjugate this peptide sequence to the chosen hydrogel or mixture hydrogels. In addition, such methods of conjugation are described for various types of hydogels by Bouhadir, K. H., et. al., (J. Polymer, 1999, 40, 3575), by Hern, D. L., et. al., (J. Biomed. Mater. Res., 1998, 39, 266), by Moghaddam, M. J., et. al., (J. Polym. Sci.: Part A: Polym. Chem., 1993, 31, 1589) and WO/2005/021580, all of which are hereby incorporated by reference in their entirety.

In some embodiments, encompassed in the methods described herein are synthetic hydrogels that are modified and/or mixed with other naturally occurring molecules to aid cell adhesion. One of ordinary skill in the art can modify synthetic hydrogels for use in the making TE constructs. Methods are also described in U.S. Pat. Nos.: 4,565,784, 5,489,261, and 7,300,962 and these are hereby incorporated by reference in their entirety.

In some embodiment, the multi-layered 3 D TE constructs are incorporated with bioactive agents. As used herein, “bioactive agents” or “bioactive materials” refer to naturally occurring biological materials found in the particular organic tissue of which the TE construct is mimicking, for example, extracellular matrix materials such as fibronectin, vitronection, and laminin; and growth factors and differentiation factors. “Bioactive agents” also refer to artificially synthesized materials, molecules or compounds that have a biological effect on the living cells that are printed and embedded within the TE construct and/or have an effect on the surrounding biological tissue at where the TE construct is implanted. For examples, peptides or recombinant vascular endothelial growth factor (VEGF) that can stimulate angiogenesis. A great number of growth factors and differentiation factors that are known in the art to stimulated cell growth and differentiation of the progenitor and stem cells. Suitable growth factors and cytokines include any cytokines or growth factors capable of stimulating, maintaining, and/or mobilizing progenitor cells. They include but not limited to stem cell factor (SCF), granulocyte-colony stimulating factor (G-CSF), granulocyte-macrophage stimulating factor (GM-CSF), stromal cell-derived factor-1, steel factor, VEGF, TGFβ, platelet derived growth factor (PDGF), angiopoeitins (Ang), epidermal growth factor (EGF), bone morphogenic protein (BMP), fibroblast growth factor (FGF), hepatocye growth factor, insulin-like growth factor (IGF-1), interleukin (IL)-3, IL-1α, IL-1β, IL-6, IL-7, IL-8, IL-11, and IL-13, colony-stimulating factors, thrombopoietin, erythropoietin, fit3-ligand, and tumor necrosis factor α (TNF-α). Other examples are described in Dijke et al., “Growth Factors for Wound Healing”, Bio/Technology, 7:793-798 (1989); Mulder G D, Haberer P A, Jeter K F, eds. Clinicians' Pocket Guide to Chronic Wound Repair. 4th ed. Springhouse, Pa.: Springhouse Corporation; 1998:85; Ziegler T. R., Pierce, G. F., and Herndon, D. N., 1997, International Symposium on Growth Factors and Wound Healing: Basic Science & Potential Clinical Applications (Boston, 1995, Serono Symposia USA), Publisher: Springer Verlag.

Examples of growth factors include EGF, bFGF, HNF, NGF, PDGF, IGF-1 and TGF-β. These growth factors can be mixed with the hydrogel precursor or mixture of hydrogels.

In some embodiments, suitable bioactive agents include but not limited to pharmaceutically active compounds, hormones, growth factors, enzymes, DNA, RNA, siRNA, viruses, proteins, lipids, pro-inflammatory molecules, antibodies, antibiotics, anti-inflammatory agents, anti-sense nucleotides and transforming nucleic acids or combinations thereof. Such suitable bioactive agents can have therapeutic effects on the tissues at the implant site and on the printed cells in the construct. For example, anti-fungal activity.

In some embodiments, the multi-layered 3D constructs described herein comprise living cells embedded within the layers of hydrogel. The living cells are printed on to partially polymerized hydrogel layers. The cell printing is computer-assist, designed to deposit the cells at specific spatial distribution on the partially polymerized hydrogel layer. A nebulized layer of cross-linking agent is then applied on top of the partially polymerized hydrogel layer having the deposited cells. This nebulized layer of cross-linking agent serves to fully polymerize the hydrogel layer having the deposited cells, thereby fully embedding and encapsulating the printed cells. In some embodiments, the multi-layered 3 D constructs described herein comprise more than one cell type embedded within the multi-layered 3 D construct. In some embodiment, more than one cell type is embedded within a single layer of the multi-layered 3 D construct, e. g. see Example 8.

In some embodiments, the cells useful for the making of the multi-layered 3-D construct described herein include but not limited to stems cells: embryonic stem cells, mesenchymal stem cells, bone-marrow derived stem cells and hematopoietic stem cells; chrondrocytes progenitor cells, pancreatic progenitor cells, myoblasts, fibroblasts, keratinocytes, neuronal cells, glial cells, astrocytes, pre-adipocytes, adipocytes, vascular endothelial cells, hair follicular stem cells, endothelial progenitor cells, mesenchymal cells, neural stem cells and smooth muscle progenitor cells.

In some embodiments, differentiated cells that have been reprogrammed into stem cells are used. For example, human skin cells reprogrammed into embryonic stem cells by the transduction of Oct3/4, Sox2, c-Myc and Klf4 (Junying Yu, et. al., 2007, Science 318: 1917-1920; Takahashi K. et. al., 2007,Cell 131: 1-12). Neural tissues were differentiated from converted skin cells.

In some embodiments, the cells useful for the 3D TE constructs are human cells. Examples include but are not limited to human cardiac myocytes-adult (HCMa), human dermal fibroblasts-fetal (HDF-f), human epidermal keratinocytes (HEK), human mesenchymal stem cells-bone marrow, human umbilical mesenchymal stem cells, human hair follicular inner root sheath cells, human umbilical vein endothelial cells (HUVEC), and human umbilical vein smooth muscle cells (HUVSMC).

In some embodiments, the cells useful for the 3D TE constructs are rat and mouse cells. Examples include but not limited to RN-h (rat neurons-hippocampal), RN-c (rat neurons-cortical), RA (rat astrocytes), rat dorsal root ganglion cells, rat neuroprogenitor cells, mouse embryonic stem cells (mESC) mouse neural precursor cells, mouse pancreatic progenitor cells mouse mesenchymal cells and mouse endodermal cells

In other embodiments, tissue culture cell lines can be used in the 3D TE constructs described herein. Examples of cell lines include but are not limited to C166 cells (embryonic day 12 mouse yolk), C6 glioma Cell line, HL1 (cardiac muscle cell line), AML12 (nontransforming hepatocytes), HeLa cells(cervical cancer cell line) and Chinese Hamster Ovary cells (CHO cells).

An ordinary skill artisan in the art can locate, isolate and expand such cells. In addition, the basic principles of cell culture and methods of locating, isolation and expansion and preparing cells for tissue engineering are described in “Culture of Cells for Tissue Engineering” Editor(s): Gordana Vunjak-Novakovic, R. Ian Freshney, 2006 John Wiley & Sons, Inc., and in “Cells for tissue engineering” by Heath C. A. (Trends in Biotechnology, 2000, 18:17-19) and these are hereby incorporated by reference in their entirety.

In one embodiment, 1×10⁴ to 1×10⁹ total cells can be delivered on a single hydrogel layer. For tissue engineered constructs, at least 1×10⁶ total cells per 1 ml volume can be delivered in suspension. Depending on the size of individual cells, cell aggregates with the density of 1×10⁸ cells per 1 ml volume can be delivered.

The inventor has printed the following cell types using the method described herein and have achieved at least over 70% and in some instances over 90% cell viability on the hydrogel layers. Human cell lines: HCMa, HDF-f, HEK, human mesenchymal stem cells-bone marrow, human umbilical mesenchymal stem cells, human hair follicular inner root sheath cells, HUVEC, HUVSMC; rat cell lines: rat neurons-hippocampal, rat neurons-cortical, rat astrocytes, rat dorsal root ganglion cells, rat neuroprogenitor cells; mouse cell lines: mESC, mouse neural precursor cells, mouse pancreatic progenitor cells, mouse mesenchymal cells, mouse endodermal cells; specialty cell lines: C166 cells, C6 glioma Cell line, HL1, AML12, HeLa and CHO cells.

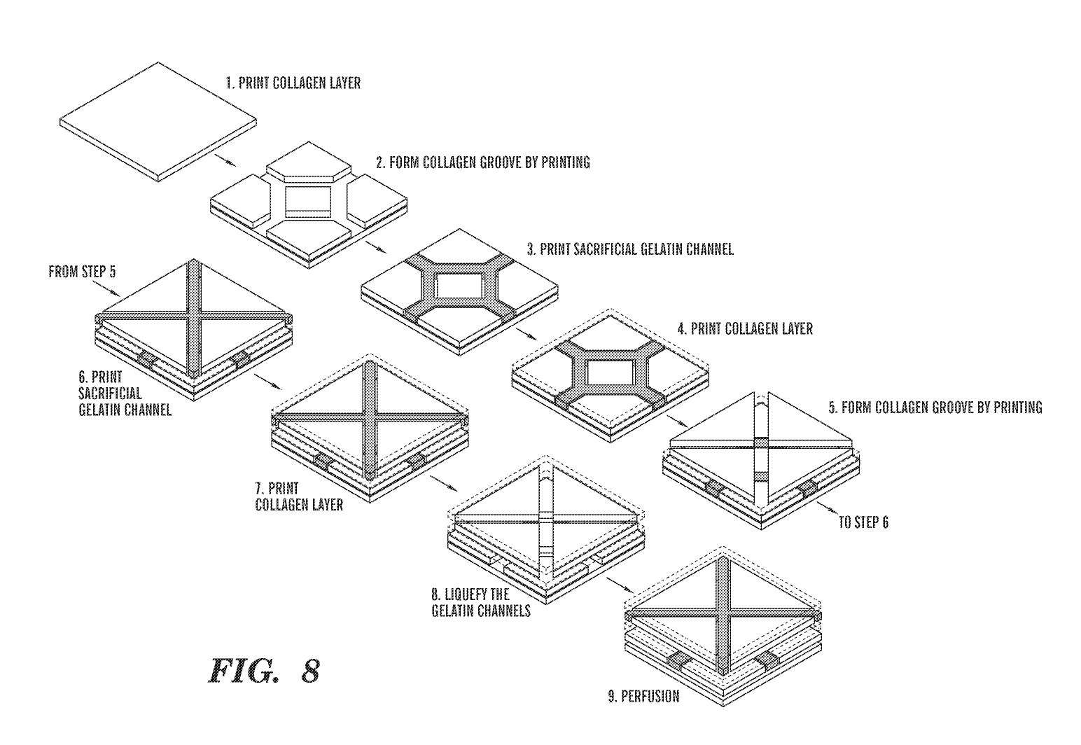

In one embodiment, the 3 D TE construct comprises channels (FIG. 8). The hydrogel precursors are printed to create the desired pattern of channels in the 3D construct (step 2, FIG. 8). The channels are filled in with on-demand printing of a low-melting point material such as gelatin (step 3, FIG. 8). Additional hydrogel layers are deposited over the layer with the channels (step 4, FIG. 8). When the 3D construct is completed, the 3D construct is heated to melt away the gelatin from the channels and the channels can be perfused with culture media, plasma, artificial blood or blood etc to nourish the cells within the construct.

In one embodiment, the 3D construct is built on a substrate. The substrate is the object/support/scaffold that is placed on the computer controlled stage in the apparatus set-up described herein on which the TE construct is built.

In one embodiment, substrate is flat. In another embodiment, the surface of the substrate is flat. In other embodiments, the substrate is non-biological, for example, made of synthetic materials. The substrates useful for the methods described herein are usually of a hydrophobic or inert nature. Examples include but not limited to polyolefins, polyurethanes, polypropylene, polyvinyl chloride, polystyten, silicone and polytetrafluoroethylene or from the group comprising medicinally acceptable metals and glass. Example of a polymeric materials polyolefins is polyethylene orpolypropylene. In one embodiment, the substrate is made of poly dimethylsiloxane (PDMS). Examples of flat surfaces include that of a polypropylene petri-dish container on the movable platform which freeform fabrication of a multi-layered 3 D TE construct can take place. In one embodiment, the first nebulized layer of cross-linking agent is applied uniformly over the flat surface of the container on which the TE construct will be built. In another embodiment, the first nebulized layer of cross-linking agent is applied to at least a portion of the substrate. Subsequently, the first hydrogel precursor is printed, via drop-by-drop on-demand printing or by continuous extrusion on to the nebulized layer of cross-linking agent, and then a second nebulized layer of cross-liking agent is applied over the hydrogel precursor layer. In some embodiments, the nebulized layer of cross-linking agent is applied over the general surface of the petri-dish container such that a nebulized layer of cross-linking agent is left covering both the areas with and without the printed hydrogel in the container. The specific shape and size of the multi-layered 3D TE construct is achieved by the computer-assisted ink-jet printing of the hydrogel precursor. In some embodiments, the first and subsequent nebulized layers are applied to the general surface of the substrate. In other embodiments, the first and subsequent nebulized layers are applied to portions of the surface of the substrate.

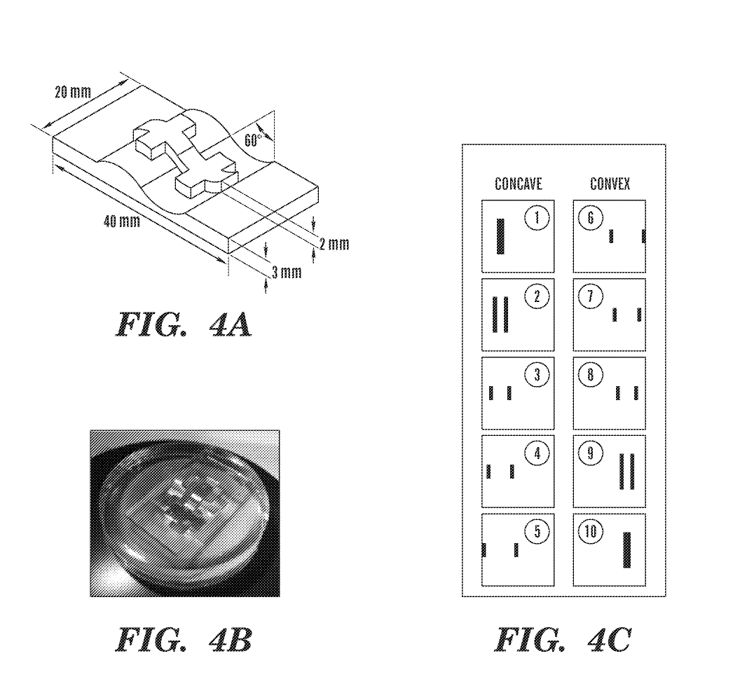

In another embodiment, the substrate is contoured, i. e. non-planar and having regions that are concave and other regions that are convex. The surface for printing is contoured, and non-planar. For example, when a multi-layered 3D TE construct is designed for a skin wound that has an uneven depth and shape. A mold can be made of poly dimethylsiloxane (PDMS) and the mold is designed to have a similar size, shape and depth to the wound. An example of a contoured PDMS mold and construction of a multi-layer 3 D TE construct is described herein. The mold is the substrate upon which the multi-layer 3 D TE construct is built. The mold is secured on to the printing platform stage of the CAD cell-hydrogel printing apparatus as described herein. The first nebulized layer of cross-linking agent is applied uniform over the contoured surface of the mold. Subsequently, the first hydrogel precursor is printed on to the nebulized layer of cross-linking agent that is found in the concave areas of the mold and then a second nebulized layer of cross-liking agent is applied over the container covering both the area with and without the printed hydrogel. Again, the specific shape and size of the construct is achieved by the computer-assisted ink-jet printing of the hydrogel precursor. When a contoured substrate is used, the hydrogel printing can be initially concentrated in the concave regions until the cavities are fully filled in by hydrogel and a planar surface has been achieved. Then hydrogel printing is uniformly applied to the planar surface of the mold in order to obtain the specific shape of the construct (see FIG. 4).

In some embodiments, the hydrogel printing is applied non-uniformly in the mold to produce a non-planar convex construct. The CAD program dictates the specific region where the layers of hydrogel are to be applied.

In other embodiments, the substrate is biological, for examples, made of extracellular matrices made naturally by living cells, a layer of cell culture on a culture dish or on a TE scaffold, or a living tissue, organ or body part.

Examples of replacement tissue that can be engineered, reconstructed and/or repaired include but not limited to craniofacial structures such as bone, adipose tissue and facial muscles, cardiac muscle, cardiac valve, skin, bones, pancreas tissue, tissue, skeletal muscles, neural tissues, diaphragmatic muscles and tendons, breast tissue, blood vessels, cartilage, tendons, ligaments, bladder, urether, uterus, ureter, virgina, cervix, trachea, hair, cornea, esophagus and small intestines. Fetal reconstructions of the tracheal and the diaphragm using tissue engineered autologous cartilage grafts and tendons respectively are fully described by Kunisaki et. al., 2006, J. Pediatr. Surg. 41:675-82 and by Fuch et. al., 2004, J. Pediatr. Surg. 39: 834-8 and these are hereby incorporated by reference.

The present invention is applicable to skin repair. Skin repair is important for the treatment of burns, lacerations and diabetic wounds. To restore the function of the skin after damage and to facilitate wound-healing process, autologous grafts are commonly used to repair the skin while avoiding immune-rejection (Ben-Bassat H, et. al., Burns, 2001, 27:425-431). However, extensive skin damage beyond the conventional graft extraction method requires rapid in vitro culture of biopsied skin cells to form a planar sheet of skin cells (Atiyeh B S, et. al., Burns 2005, 31:944-956; Wood F M, et. al., Burns 2006, 32:395-401; MacNeil S. Nature 2007, 445:874-880). These sheets are transplanted back to the wound site to prevent fluid loss and infection while promoting skin repair process. Using the invention described herein, a mold of the wound is made, then a custom made multi-layered 3D skin construct can be prepared on a substrate made from the mold. The custom made multi-layered 3D skin construct can be prepared in a short period of time, within the same day of injury, and the construct will correspond to the size and depth of the wound.

In order to address deeper skin damage involving dermal layers under the basal lamina, several techniques have been developed by combining biocompatible materials with key cellular components of skin grafts. For example, dermal cellular components such as fibroblasts are combined with a biomaterial matrix, such as a silicone-based sheet (Burke J F, et. al., Ann Surg. 1981, 194:413-428), to stimulate cellular redevelopment and vascularization at the wound site (Cuono C., et. al., Lancet 1986, 1:1123-1124; Stern R, et. al., J Burn Care Rehabil. 1990, 11:7-13). Autologous keratinocytes have also been integrated with a compatible xenotransplant of bovine collagen to assist the regeneration of both dermal/epidermal skin layers (Boyce S T, et. al., Ann. Surg., 1995, 222:743-752; Boyce ST, et. al., Ann. Surg., 2002, 235:269-279; Supp DM and Boyce S T., Clin. Dermatol. 2005, 23:403-412).

Three-dimensional (3D) organotypic reconstruction of the multiple skin layers have been proposed for skin repair (Boyce S T, et. al., Ann. Surg., 1995, 222:743-752; Ralston D R, et. al., Br J Dermatol. 1999, 140:605-615; Sahota P S, et. al., Wound Repair Regen. 2003, 11:275-284; Sun T, et. al., Tissue Eng., 2005, 11:1023-1033) and for modeling the progresses of skin diseases or damages (Barker C L, et. al., J Invest Dermatol 2004, 123:892-901; Eves P, et. al., Clin. Exp. Metastasis, 2003, 20:685-700). Stratified skin cellular structure is a crucial for the regeneration of the cell-to-cell, or cell-to-extracellular matrix interactions, which are necessary for normal skin function. To artificially construct stratified layers of skin cells, dermal fibroblasts are seeded in a collagen scaffold below epidermal keratinocytes (Gangatirkar P, et. al., Nat. Protoc. 2007, 2:178-186). However, in case where the organotypic skin culture is needed for the purpose of wound repair, 3D morphology of the skin construct, specifically tailored to the patient's wound site, cannot be readily generated via manual seeding of skin cells into hydrogel scaffold.

The methods described herein allows for the construction of an artificial tissue, either autologous or non-autologous in nature, by printing cells and natural/synthetic biomaterials in strategic locations with the help of high-precision robot. The replacement skin tissue is custom-designing of the shape, size and depth of the skin wound. The printed tissue construct can be introduced to the target area wound area after a period in vitro culture.

In the examples described herein, a stratified skin cell layers was constructed in 3D via robotic cell printing technique using an established in vitro human dermal/epidermal skin model. The printing of the multi-layered skin cells was on the poly(dimethylsiloxane) (PDMS) mold that mimics a skin wound with a 3D surface contour. The stratified layers of printed fibroblasts and keratinocytes were confirmed through immuno-fluorescence confocal imaging. The morphological information of the tissue composite (to be printed) was converted to 2D planar information and later used to dictate printing motions for on-demand construction of the skin layers. Unlike the existing multi-layered printing methods which require the planar target surface, in the methods described herein, a layer of cell-containing collagen precursor is printed via non-contact type micro-dispenser, and subsequently cross-linked by coating of the nebulized aqueous cross-linking agents (sodium bicarbonate). It eliminated the needs of having separate planar containers for cross-linking agents and enabled direct, on-demand fabrication of the 3D tissue composites on non-planar surfaces.

### Definitions of Terms

As used herein, the term “nebulization” refers to conversion into an aerosol or spray.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

The term “hydrogel” refers to a broad class of polymeric materials which are swollen extensively in water but which do not dissolve in water. Generally, hydrogels are formed by polymerizing a hydrophilic monomer in an aqueous solution under conditions where the polymer becomes cross-linked so that a three-dimensional polymer network sufficient to gel the solution is formed. Hydrogels are described in more detail in Hoffman, A. S., “Polymers in Medicine and Surgery,” Plenum Press, New York, pp 33-44 (1974).

As used herein, the term “tissue engineered composites” refer to tissue engineered constructs that are made of two or more constituent materials with significantly different physical or chemical properties and which remain separate and distinct on a macroscopic level within the finished structure. For example, a TE composite described herein is made of hydrogel-collagen and living cells, or collagen, fibrin, and living cells. The term ‘composite” and “construct” are used interchangeably.

As used herein, the term “on-demand” refers to the operator control over the printing of the hydrogel in freeform fabrication.

As used herein, the term “substrate” refers the surface and material upon which the TE construct is to be built. The substrate is the object/support/scaffold that is placed on the stage in the apparatus set-up on which the TE construct will be built. The substrate can be a synthetic object such as a petri-dish, in which case the substrate is non-biological. The substrate can be also be a piece of living tissue, for example, one with damage and need repair, in which case the substrate is biological.

As used herein, the term “non-biological” refers to a substrate that is comprised solely of synthetic materials. “Non-biological” also refers to not involving, relating to, or derived from biology or living organisms.

As used herein, the term “biological refers to a substrate that is comprised of materials that involves, relate to, or are derived from biology or living organisms. For example, extracellular matrices made naturally by living cells, a layer of cells cultured on a culture dish or on a TE scaffold, or a living tissue, organ or body part.

As used herein, the term “channel” in a 3 D TE construct refers to a tube-like passage way that connects different parts of the construct. This “channel” is not filled with cross-linked hydrogel material. Instead, the passage way is hollow to allow fluidic material to flow through.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages may mean±1%.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The present invention can be defined by any of the following alphabetized paragraphs:
[A] A method of making a three dimensional multi-layered hydrogel construct, the method comprising the steps of: (a) applying a first nebulized layer of cross-linking material on a substrate;(b) depositing at least one layer of hydrogel precursor on top of the first nebulized layer of cross-linking material, wherein the hydrogel precursor cross-links upon contact with the nebulized layer of cross-linking material to form a partially cross-linked gel; (c) applying a second nebulized layer of cross-linking material on top of the partially cross-linked gel of step (b), thereby promoting completing cross-linking of the layer of hydrogel of step (b); and (d) repeating alternating step b followed by step (c).
[B] The method of paragraph [A], wherein the hydrogel layer is deposited via drop by drop on-demand printing or continuous extrusion of the precursors.
[C] The method of paragraph [A], wherein the nebulized cross-linking material comprises 1-100 micrometer sized droplets.
[D] The method of paragraph [A], wherein step (d) is repeated 1-20 times.
[E] The method of paragraph [A], wherein step (d) is repeated at least 5 times.
[F] The method of paragraph [A], wherein step (d) is repeated at least 10 times.
[G] The method of paragraph [A], wherein step (d) is repeated at least 15 times.
[H] The method of any of paragraphs [A]-[G], wherein the multi-layered three dimensional construct comprises more than one type of hydrogel.
[I] The method of any of paragraphs [A]-[H], wherein the hydrogel precursor is selected from a group consisting of collagen, gelatin, fibrinogen, chitosan, hyaluronan acid, alginate, poly-ethylene glycol, lactic acid, and N-isopropyl acrylamide.
[J] The method of any of paragraphs [A]-[I], further comprising depositing living cells on the layer of hydrogel precursor after step (b) but prior to step (c).
[K] The method of paragraph [J], wherein more than one cell type is deposited in the multi-layered three dimensional construct.
[L] The method of paragraph [K], wherein the cell types are selected from a group consisting of stems cells, pancreatic progenitor cells, neuronal cells, vascular endothelial cells, hair cells, mesenchymal cells, and smooth muscle cells.
[M] The method of paragraph [B], wherein the substrate is flat.
[N] The method of paragraph [B], wherein the substrate is contoured.
[O] The method of paragraph [B], wherein the substrate is biological.
[P] The method of paragraph [B], wherein the substrate is non-biological.
[Q] The method of any of paragraphs [A]-[P] wherein the three dimensional multi-layered hydrogel construct further comprise of channels.
[R] A three dimensional multi-layered hydrogel construct comprising at least 10 layers of hydrogel material, at least one type of cells, wherein the cells are deposited on different layers of hydrogel material, and at least one type of hydrogel material.
[S] The three dimensional multi-layered hydrogel construct of paragraph [R] wherein the cells types are fibroblast and keratinocytes.
[T] The three dimensional multi-layered hydrogel construct of paragraph [S] further comprising hair follicular stem cells.
[U] The three dimensional multi-layered hydrogel construct of paragraph [R] wherein the cells types are vascular endothelial progenitor cells and smooth muscle progenitor cells.
[V] The three dimensional multi-layered hydrogel construct of paragraph [R], wherein the cells types are pancreatic endothelial progenitor cells and mesenchymal cells.
[W] The three dimensional multi-layered hydrogel construct of paragraph [R], wherein the cells types are neurons and astrocytes.
[X] The three dimensional multi-layered hydrogel construct of paragraph [R], wherein the cells types are neural stem cells and astrocytes.
[Y] The three dimensional multi-layered hydrogel constructs of any of paragraphs [R]-[X], further comprising bioactive agents.
[Z] The three dimensional multi-layered hydrogel construct of any of paragraphs [R]-[Y], wherein the cells are deposited on different layers of hydrogel material.

This invention is further illustrated by the following example which should not be construed as limiting. The contents of all references cited throughout this application, as well as the figures and table are incorporated herein by reference.

### EXAMPLES

### Materials and Methods

### Designing and Arrangement of the Three-Dimensional Cell-Hydrogel Printer.

The overall schematic of the printing hardware comprises a modular cell printing platform having fluid cartridges for cells and hydrogel precursors; a dispenser array; target substrate; horizontal stage; vertical stage; range finder; vertical stage heater/cooler; optional independent heating/cooling unit for the dispenser. A 4-channel dispenser array is the typical design. The printer consists of modules of 4-channel array microvalves (SMLD Fritz Gyger AG, Thun-Gwatt, Switzerland) and a three-axis Cartesian robotic stage that control the timing and location of dispensing of cells in suspension and collagen precursors. The dispensing array, with a pneumatically-driven control mechanism (shown in the later section), is mounted to the horizontal (x-y) robotic stage (Newmarksystems, CA; with bidirectional reproducibility of 5 μm). The target substrate is mounted to another robotic stage that moves along the vertical direction (z-axis). The cell suspension in culture media and hydrogel precursors in aqueous form are placed in disposable plastic syringes (equivalent to the ink cartridges in commercial printer) and continuously fed to the dispensing array under pneumatic pressure. The entire device is housed in a laminar flow hood (StreamLine, FL) with two cameras (Pixelink PL-A741, Ottawa, Canada and, UBV-49, Logitech, CA) use for (1) measuring the droplet size and for (2) visual inspection of tissue engineered constructs. The dispensers and target substrates are temperature controlled (at 20° C., operating temperature between 5° C. to 40° C.) by solid-state thermoelectric device (TED; TE Technology, Traverse City, Mich.). All cell/solution compartments and tubings used in the experiments are disposable and replaceable. All the machine parts are designed in detachable modules for easy assembly and modification.

### Software Interface and Hardware Implementation

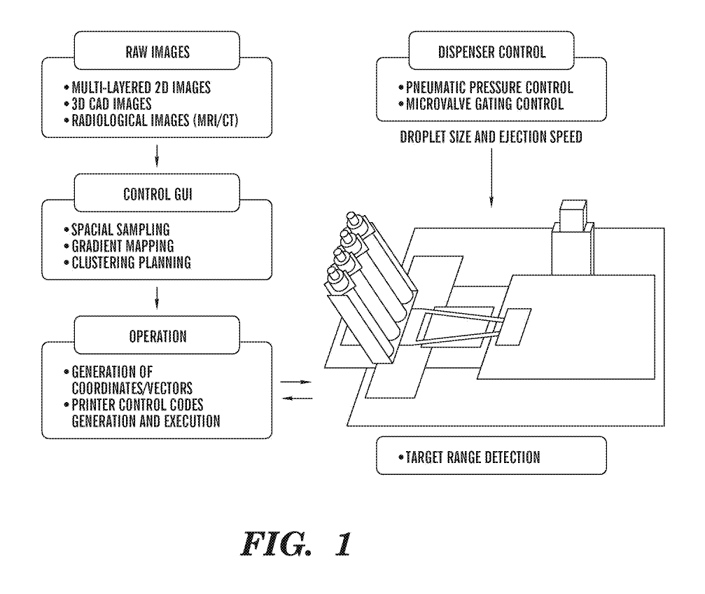

The schematic of the user-interface for the printer is shown in FIG. 1. The MATLAB computation environment (Mathworks, Natick, Mass.) is used to generate the robot control codes dictating the dispensing spatial coordinates. Information on the target substrates, as input to the printer, is prepared from a slice-by-slice profile of the images representing the desired structure or from digital photographic images. Optionally, 3D computer-aided-design (CAD) files (SolidWorks, Concord, Mass.) or slice-by-slice 3D radiological images (for example, from MRI or CT) can be used as input files. For example, 2D information representing the sections of a 3D object can be obtained via virtually ‘slicing’ the volume through interpolation routines such as nearest neighbor or tri-linear methods (Sun W, et. al., Biotechnol. Appl. Biochem. 2004, 39:29-47; Hill DL, et al., Phys. Med. Biol., 2001, 46:R1-45). The dispensing coordinates are then spatially sampled from the 2D sectional images. The distance between the each dispensing points (thus printing resolution) along with the desired printing dimension is user-definable. The sampled printing coordinates are routed to the path planner algorithm (either through vector or coordinate-by-coordinate mode), which prescribes the printing sequence. The path can be defined in either sequential line printing or boundary-printing followed by sequential filling. This process is similar to the printing routine for many types of commercial plotters. Spatial gradient of dispensing density as well as the clustering of dispensing sequence, in order to save printing time, can be implemented, with optional 3D ‘preview’ function to help the user to plan/monitor the printing process. The generated control codes are sequentially executed by scripts generated by Active-X Toolkit (Galil Motion Control, Inc., Rocklin, Calif.) programmed in Visual Basic (Microsoft, Redmond, Wash.). An ultrasonic range finder (SRF04, Devantech, Norfolk, UK) is used to maintain the distance between the dispenser nozzle and target substrates by adjusting the movement of a vertical stage. The volume of droplet is changed independently across all four channels of dispensers by controlling the pneumatic pressure to the fluid paths or by controlling the opening duration for the microvalve. Other extracellular matrix materials and/or bioactive agents such as cytokines can be prepared as liquid, dispensed and integrated into hydrogel during sequential dispensing.

### Control of Fluid Dispensing

The general operating principle of the dispensing mechanism is described herein. Cell suspension and uncross-linked hydrogel precursor are placed in 5 or 10 mL disposable syringes. Each syringe is independently pressurized using an air tank (filtered with 0.2 μm porosity where appropriate) via a digital pressure regulator (ITV-2010, SMC, Japan). The fluidic pathway from the syringe, under the pneumatic pressure, is gated by a set of electromechanical microvalves (150 μm nozzle diameters) using a standard TTL pulse (Electromechanica, East Freetown, MA). With the minimal open/close duration of valve is 200 μs, the maximum duty cycle allowed is at least 1000 Hz of dispensing. The advantage of using a pneumatically-driven electromechanical valve is that various types of liquid materials with different viscosities (up to 200 Pa·s) can be dispensed by adjusting the pressure and valve gating time. Based on the Bernoulli's principle on fluids, the droplet ejection speed is controlled by regulating the pressure. The shock during surface impact is less of a concern for cell viability since the printed cells are cushioned by the partially polymerized hydrogel bed at typically low ejection velocity less than 3 m/s (as measured by the on-board high-speed camera). Unlike the potential pressure-related cell damage which can occur in ink-jet or piezoelectric element-driven dispensing, high cell viability is achieved due to the low operational pneumatic pressure (on the order of 1-3 pounds per square inch-psi).

### Electromechanical Dispenser Array

Commercial ink-jet based dispensing devices, based on either bubble jet or piezoelectric elements, can be pre-calibrated for dispensing ink with a fixed degree of viscosity, but do not provide flexibility in printing hydrogel materials with different viscosities. In addition, a small nozzle diameter often limits the size of the cells to be printed. Miniature electromechanical valves allow for the dispensing of a wide range of low viscosity liquids less than 200 centipoise (cP). With a nozzle diameter of 150 μm, the valve accommodates the dispensing of larger cells, which are unable to be printed using commercial ink-jet printers. The advantage of the pneumatically-driven continuous dispensing method includes the ability to control the volume of droplets by changing either the pressure to fluid pathway or the duration of the valve opening time.

The liquid state hydrogel precursors and suspended cells were kept in disposable 10 mL syringes and pressurized with HEPA filtered ambient air. The pneumatic pressure to the liquid was regulated by a digitally-controlled pressure regulator (ITV2010, SMC, Japan) and the duration of the valve opening was controlled by changing duration of standard TTL pulse (>150 μsec). To dispense the gelatin as a liquid substance, one of the dispensers and syringe reservoirs (5 mL) was enclosed in aluminum housing and heated to 40° C. by a temperature regulated thermo-electric device (TED, also known as Peltier device, TE Technology, Traverse City, Mich.). A total of three microvalves were used for dispensing collagen, gelatin, and hFB suspension.

The volume of the dispensed droplet size of 7% (w/v) gelatin solution in distilled water was characterized in terms of applied pneumatic pressure and valve gating period at 40° C. The pneumatic pressure was varied from 6 psi to 13 psi (with a step of 1 psi), and valve gating time was adjusted among 450 μs, 600 μs, and 750 μs. The size of the dispensed gelatin droplet was directly measured by imaging the droplet in the midair at room temperature (20° C.) by a high-speed camera (Pixelink PL-A741, Ottawa, Canada) by synchronizing the image acquisition upon dispensing (shutter speed=20 μs).

### Preparation and Printing of Collagen Hydrogel Precursor

Type I collagen (rat tail origin; BD Biosciences, MA) is used as hydrogel precursor for a scaffold material. First, the collagen precursor is diluted to 2.05 mg/mL with 1× Dulbecco's phosphate-buffered saline (DPBS) and kept on ice. The pH of the diluted collagen was approximately 4.5, and the precursor remains uncross-linked, to be dispensable by the micro valve dispenser. After sterilizing the syringes and fluidic pathways, the prepared collagen precursor solution is loaded into a syringe and subsequently printed to fill 10 by 10 mm square area using the inter-dispensing distance (spatial resolution) of 600 μm. The droplets of collagen precursor are printed with pressure of 2 psi with a valve opening time of 600 μs.

Chemically-crosslinkable (solution-phase in acidic pH and gel-phase in neutral pH) collagen hydrogel precursor (rat tail, type I, BD Bioscience, MA) and temperature-sensitive gelatin (Porcine skin Type A, SIGMA ALDRICH®) were used for the construction of the hydrogel scaffold with fluidic channel. Sodium bicarbonate (NaHCO₃) in distilled water (0.8 M) was used as a crosslinking material for the collagen hydrogel precursor (Gangatirkar et al. 2007). A 7% (weight/volume; w/v in distilled water) gelatin solution was prepared at 40° C. before printing and then loaded into a heated dispenser unit.

For bioprinting of neuronal cells, the collagen precursor was diluted to 1.12 mg/ml with 0.02 N acetic acid solution and 1× phosphate-buffered saline (Gibco, New York, USA) (volume ratio of 1 :1:2; final pH=4.5) and kept on ice. A degree of optimization of collagen scaffold concentration is needed to ensure the proper neurite outgrowth while preserving the mechanical integrity of the scaffold itself. The dilution factor was determined from the collagen density that showed the most significant neurite outgrowth among three different densities of collagen (2.23, 1.49 and, 1.12 mg/ml). A collagen concentration lower than 1.12 mg/ml affected the integrity of the collagen gel in the media as the collagen hydrogel partially dissolved into the media.

For bioprinting of a collagen-fibrin scaffold, the collagen, in an uncrosslinked liquid form, was diluted to concentrations of 1.74 mg/mL and 1.16 mg/mL with 1× phosphate buffered saline (PBS, pH 7.4; GIBCO®) at 4° C. and placed on ice until loaded into the syringe for printing. A concentration of 0.87 mg/mL was also prepared by diluting the collagen precursor with 1× PBS and 0.02 M acetic acid (collagen:PBS:acetic acid, 1:2:1) to maintain a pH of about 4 to keep collagen uncrosslinked during printing. These three different concentrations of collagen were tested to maximize the cell proliferation and migration while preserving the mechanical integrity of the printed collagen scaffold.

### VEGF Containing Fibrin Gel Preparation for Printing

The fibrin gel was created by combining solutions containing fibrinogen, thrombin, and heparin according to prior work (Jeon et al., 2005, J. Control Release 105:249-259). Because these components form a gel when mixed together (gels could not be printed as individual droplets), the aqueous solutions containing the components were prepared into two separate printing cartridges. The fibrinogen solution was prepared by diluting the fibrinogen (type IV from bovine plasma; SIGMA-ALDRICH®) to 62.8 mg/mL using PBS and was mixed with 132 U/mL aprotinin in PBS. Aprotinin was used as an enzyme inhibitor to prevent fibrin degradation. The thrombin solution was prepared by combining thrombin, heparin, and calcium chloride (CaCl₂) (all from SIGMA-ALDRICH®) in PBS. Heparin promotes neurite extension of printed NSC (Sakiyama et al., 1999, J. Control Release 69:149-158) while CaCl₂ was added to preserve the integrity of fibrin (Bhang et al., 2007, J. Biomed. Mater. Res. A 80:998-1002). The concentrations of these components in the solution were 133.2 NIH U/mL for thrombin, 4.76 μg/μL for heparin, and 11.8 mg/mL for CaCl₂.

VEGF (SIGMA-ALDRICH®) stock solution was prepared by the dilution of the VEGF with distilled water to 0.1 mg/mL, according to the manufacturers' directions. Subsequently, the VEGF stock solution (5 μL) was mixed (1:1, volume:volume) with 5 μL thrombin or 5 μL fibrinogen solutions.

### Cell Culture and Preparation for Printing

Primary adult human dermal fibroblasts (hFB) and primary adult human epidermal keratinocytes (hKC) are purchased from ScienCell Laboratory and cultured in standard condition of 37° C., 5% CO₂, 2% fetal bovine serum. 1% FB growth supplements was added to FB media while 1% KC growth supplements were added to KC media. 1% penicillin-streptomycin (SIGMA-ALDRICH®) is also added to both culture media. Culture media are changed every other day.

Both hFB and hKC cell lines were subcultured when cells are grown to 70% confluency. hFB and hKC are used for printing experiments at passage number 6. The harvested cells are suspended in the required culture medium at a concentration of 1×10⁶ cells/mL. Cell suspensions with lower concentration (<10⁵ cells/mL) do not promoted the proliferation of the printed cells, and those in higher concentration than 3.0×10⁶ cells/mL induced clogging problems in tubes and dispenser with aggregated cell pellets. The syringes and fluidic pathways for cell printing are sterilized with 70% alcohol, flushed with endotoxin-free, distilled water and dried via HEPA filtered air. And then the cell suspensions were loaded in the syringes of the cell printer, and gently vibrated during printing experiments to prevent cell aggregation. The droplets of cell suspension were printed with pressure of 1.2 psi with a valve opening time of 500 μs.

Astrocytes and neurons from embryonic rat (day 18; BrainBits LLC) were prepared according to the vendor's protocol. The neurons were suspended in the media and loaded into the syringe after dilution to a concentration of 3×10⁶cells/ml. The astrocytes were subcultured (passage 3) and loaded intoanother syringe at a concentration of 1×10⁶cells/ml.

A concentration of astrocyte and neuron cells greater than 5×10⁶cells/ml was avoided owing to the cell aggregation and the potential clogging of the dispensing nozzle. The syringes containing the neural cell suspensions were gently vibrated to prevent cell aggregation. The viability of the printed neural cells was first examined. Neural cells were printed directly onto a 96 multiwell plate coated with poly-D-lysine (SIGMA-ALDRICH®). As a control, the cells were manually plate

The murine neural stem cell (NSC) line C17.2 was used for cell printing (Snyder et al., 1992, Cell 68.33-51). Dulbecco's modified Eagle's medium (DMEM) containing L-glutamine and 4.5 g/L of D-glucose was used to culture the NSCs. 10% fetal bovine serum, 5% horse serum, 100 U/ml penicillin, and 100 μg/ml streptomycin were added to the media (all from INVITROGEN® Inc.). The cells were grown in T-75 flasks for approximately 5 to 7 days to ≧80% confluency prior to harvesting. The cells were kept within 3 passages for all the experiments. For use in printing, the cells were trypsinized for 3 minutes at 37° C. by using Trypsin-EDTA (2.5 g/L Trypsin, 0.38 g/L EDTA) after rinsing with Dulbecco's phosphate buffer saline (DPBS; ScienCell Research Laboratories), and resuspended in the growth medium at a concentration of 1×10⁶ cells/mL upon centrifuging at 1000 rpm for 3 minutes.

### Live/Dead Staining for Viability/Cyto-Toxicity Test of Dispensed Cells

A cell viability assay is performed for printed cells 3 h after dispensing using a commercially available live/dead assay kit (Molecular Probes, MA). A group of unprinted cells is separately prepared as a control group. The samples are rinsed with DPBS, and incubated for 40 min in a solution of 5 μL of calcein AM and 20 μL of ethidium homodimer-1 in 10 mL of DPBS (dead cells show up as red fluorescence and live cells show up as green fluorescence). Cellular fluorescence was observed in an inverted epifluorescent microscope (Olympus USA, Melville, N.Y.) using a FITC/RhoA band filters.

For C17.2 (murine neural stem cells) cell viability test, the C17.2 cells were printed in the center of scaffolds in a square pattern (5×5 mm²) with a resolution of 700 μm (p=1.1 psi, Δ=500 μs). The scaffolds were of three different collagen concentrations printed on a 10 ×10 mm² area at a printing resolution of 500 μm (p=2.2 psi, Δ=500 μs) for determining the optimal for C17.2 cells to survive, proliferate, and differentiate in the scaffold.

### Immunohistochemistry (IHC) for Immunostaining

β-tubulin (CYTOSKELETON, Cell Signaling Technology, Inc.) and pan-keratin (keratin, Cell Signaling Technology, Inc.) were used to label key cellular features of both hFB and hKC that are printed tissue culture dish. The main differentiating cell label was anticipated as keratin in which hKC have an abundant source while hFB lacks the presence of keratin. The printed multi-layered cell-collagen composites cultured for 4 days were rinsed in 1× phosphate-buffered saline (PBS), fixed with 4% formaldehyde for 15 min, and rinsed three times in 1× PBS for 5 min each. After incubating in the blocking solution (5% normal mouse serum and 5% normal rabbit serum prepared in PBS with Triton X-100) for 60 min at room temperature, printed cell-collagen composites were exposed to pan-keratin (C11) mouse monoclonal antibody and β-tubulin (9F3) rabbit monoclonal antibody diluted in 1× PBS (tubulin=1:200 in PBS; keratin=1:200 in PBS) overnight at 4° C. Subsequently, fluorescence-labeled secondary antibodies were applied. The 3D architecture of stained samples was visualized using a Nikon C1 confocal system.

### Example 1

### Constructing Multi-layered Cell-Hydrogel Composites

To construct multi-layered cell-hydrogel composites, the dispensed hydrogel precursors (in a liquid state) must be cross-linked to form a hydrogel layer before printing any subsequent layers (wherein cells can be present or absent). The dispensing of hydrogel precursors and cross-linking agents on the same location, as a liquid droplet, does not generate the desired printing pattern since two liquid drops, when placed in proximity, immediately form a single drop due to the surface tension; thereby distorting the intended morphology of the tissue constructs. The problem worsens when large size of droplets (depending on the viscosity of the material, in the order of exceeding 100 μm is diameter) is used for patterning. The solution designed by Boland and colleagues (Biotechnology journal 2006, 1:910-917) is to ‘dip’ the printed hydrogel pattern (sodium alginate) into the cross-linking solution (containing calcium chloride) to make a 3D hydrogel structure. More recently, Chang and colleagues (Tissue Eng Part A, 2008,14:41-48) proposed the extrusion of viscous hydrogel precursor (sodium alginate) as a continuous strand onto the bed of cross-linking solution (aqueous calcium chloride) to form 3D micro-organ. However, these methods require a separate container to prepare a leveled planar surface of hydrogel/cross-linking materials. In addition, optimization of the concentrations hydrogel precursor and cross-linking material is required for the proper spatial patterning along with the risk of ‘washing-away’ the printed product during the dipping process.

To overcome this limitation, a new method to construct 3D hydrogel composites is described herein. As illustrated in FIG. 2, first, the substrate surface [1] is coated with cross-linking agent [2], in this case, a sodium bicarbonate (NaHCO₃) solution (0.8 M concentration in distilled water) nebulized via an ultrasonic transducer (14 mm in diameter operating at 2.5 MHz resonance frequency) (see FIG. 2 step 2). The collagen layer [3] is then printed on the coated surface of cross-linking agent [2] (see FIG. 2 step 3). During this process, the generation of ultra-fine mists about 2 μm in diameter is crucial to cross-link the dispensed collagen precursors without macroscopically distorting the printing morphology. Printed collagen droplets [3], due to a larger volume compared to the nebulized cross-linking agent, immediately cross-linked to form a gel while conserving printed morphology of the printed droplets. The size of the dispensed hydrogel droplet is in the order of 200-300 μm in diameter when landed on the nebulized layer of NaHCO₃. During the printing of cells, the droplets of cell suspension [4] in culture media were dispensed on the partially-cross-linked hydrogel layer [3] so that the cells will be lodged inside the hydrogel layer. A second layer of NaHCO₃ solution [5], again in nebulized form, was then applied on the surface of the hydrogel [4] to cross-link the remainder of the collagen layer. The top surface of this second layer of NaHCO3 [5] served as the cross-linking materials for the next hydrogen layer to be printed. The process was repeated to construct multiple layers of collagen and cells. Consequently, the multi-layered fabrication can be conducted on non-planar surfaces without the preparation of a separate container for cross-linking materials. The constructed multi-layered cell-hydrogel composites were incubated in 37° C., 5% CO₂ for 20 min before the culture media was added.

### On-Demand Planar Multi-layer Cell-Hydrogel Printing

Using the method described herein to enable construction of multi-layer cell-collagen composites, a total of 10 layers of collagen were sequentially printed on planar square in a 60 mm tissue culture dish (FIG. 3). Human dermal fibroblast (hFB) and keratinocyte (hKC) layers are located in the second and the eighth layer of collagen hydrogel (counted from the bottom layer), respectively. Five layers of collagen are sandwiched between the layers of hFB and hKC to demonstrate the ability to print spatially-distinctive cell layers. Upon printing, the cell-collagen composites are cultured in 37° C., 5% CO₂ in KC media. The medium is changed every other day.

### Droplet Size, Cells per Droplet, and Viability Assay of Printed Skin Cells

The dispensed droplet volume of cell-containing media is approximately 23 nl, when measured at the pressure of 1.2 psi with microvalve opening time of 500 μs. Further analysis showed that dispense volume of cell suspension was 8.1±2.1 nl, when measured at the pressure of 1 psi with microvalve opening time of 450 ps. The volume of collagen precursor at given dispensing condition (2 psi with valve opening time of 600 μs) is 7.63±2.73 nl (n=5). At tested concentrations of cell suspensions is about 10⁶ cells/mL for both cells, the number of cells contained in each droplet is measured to be 93±13 cells/droplet for hFB and 68±13 cells/droplet for hKC (n=36). The number of cells contained in each droplet is several times larger than the theoretical calculation (23 cells/droplet at the given cell suspension density).

The morphology of the printed cells is monitored after day 1 of culture. There is no morphological difference observed for both of hFB and hKC when compared to manually plated cells. The viability of control hFB is 96.6±3.9% while printed hFB has a viability of 95.0±2.3% (n=30). The viability of control KC was 83.9±7.1% and that of printed hKC was 85.5±5.7% (n=30). There is no significant difference in viability of printed hFB and hKC compared to each control group (p>0.05; t-test two-tailed), indicating that the cell dispensing method did not affect the cell viability.

### Example 2

### Testing of Printing Resolutions and Patterning

Prior to 3D multi-layered cell-hydrogel printing, the growth tendencies of printed hFB in the collagen hydrogel are monitored through bright field microscopy. Six different printing resolutions (in terms of inter-dispensing distance) of 200, 300, 400, 500, 700 and 900 μm are examined for printing hFB in the collagen hydrogel. The hFB suspension (concentration of 1×10⁶ cells/mL) is printed in the upper layer of two collagen layers and the growth tendency is monitored on culture day of 1 and 8. With printing resolution of 300 μm, the hFB reached cell confluency within 10 days after printing; therefore, the printing resolution of 300 μm is selected for subsequent 3D printing experiments. To confirm the reliability of on-demand 2D printing, a ‘plus’ shaped hFB pattern, consisting of 5 mm length of vertical and horizontal lines, is printed.

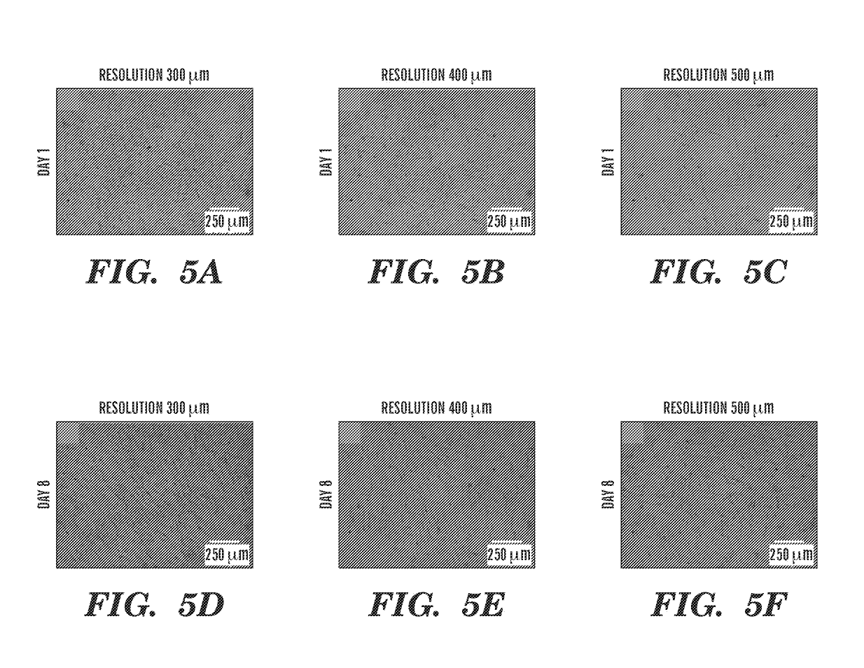

The hFBs printed at different spatial resolutions were observed under bright field microscope. The hFB printed in low printing resolution (700 and 900 μm; data not shown) did not reach sufficient cell growth in 7 days. The attempt to print the cells in high (200 μm) resolution resulted in failure of proper encapsulation in collagen bed due to the excessive amount of media compared to the printed collagen material. Day 1 culture images of FIGS. 5A-C show the cell density difference among the three groups with different printing resolutions (300, 400, and 500 μm inter-dispensing distance). The sparsity of the cells by adjusting the printing resolution is apparent from the pictures that were taken on Day 1. After 8 days of culture, printed hFB with 300 μm resolution showed the highest cell density when compared to the other groups. A similar cell density was shown between printed hFB in 400 μm resolution and those in 500 μm resolution. In the culture of hFB in collagen hydrogel, texture pattern of hFB growth was observed, and the hFB printed in higher resolution (300 μm) showed the texture pattern first (FIG. 5D). FIG. 5G shows the 2D printing of a plus shape hFB pattern imaged on Day 1. After 7 days of culture, the pattern could no longer be identified due to excessive cell proliferation (data not shown).

### Example 3

### On-Demand Planar Multi-Layer Printing of hFB and hKC

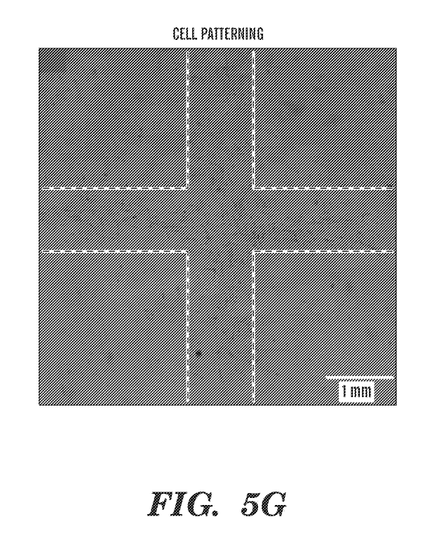

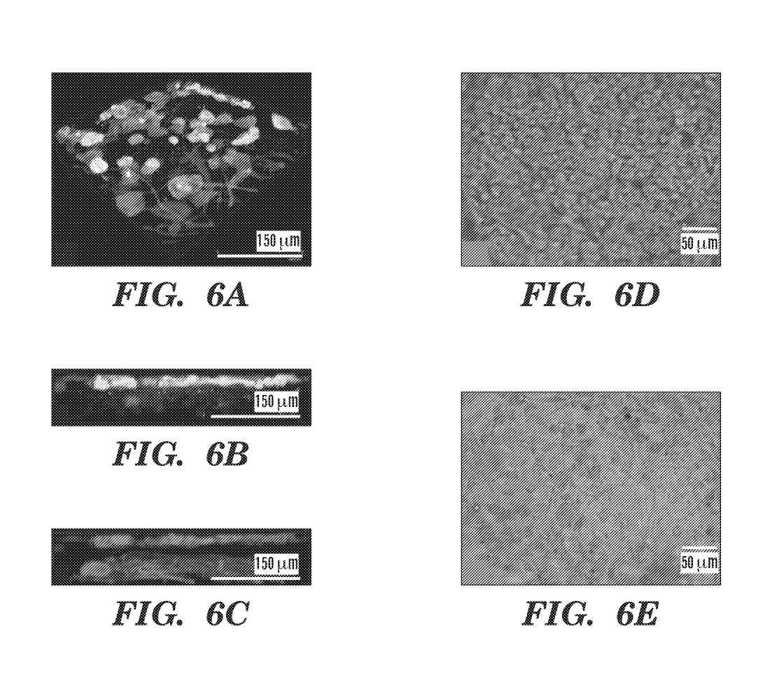

FIGS. 6A-C show confocal microscope images of printed multi-layer hFB and hKC at Day 4 of culture after immunostaining. Imaging software (Nikon EX-C 1) was used to alternate the presence of each fluorescent dye in the image (FIG. 6A with volume rendered sample). Nuclei, keratin and β-tubulin were differently labeled. FIG. 6B shows the keratin-containing hKC layer with spherical morphology. FIG. 6C (labeling for β-tubulin) illustrates both bottom and upper cell layers contain β-tubulin. hFB layer, approximately 100 μm below the surface of the culture media, shows extensive tree-like morphology which is common in a 3D culture environment (Toriseva M J, et. al., J Invest Dermatol., 2007, 127:49-59). The clear distinctive layers of hFB and hKC were visible under the projection images in FIG. 6B and C. The inter layer distance of approximately 75 μm was observed, indicating that each collagen layer occupied about 15-25 μm (5 layers of collagen were included between the hFB and hKC layers). FIG. 6D and 6E show the bright field images of hKC and hFB layers after 3 days in culture, respectively.

### Example 4

### A PDMS Mold of 3D Skin Wound Model

A PDMS mold, which simulates a shape of non-planar skin wound, was constructed to examine the ability to directly print multi-layered cell-collagen composites on 3D contoured, non-planar surface. PDMS is biologically-inert and provide excellent optical transparency for observing the printed cell-hydrogel composites on it. To construct the PDMS mold, first, an aluminum cast was prepared to imprint a negative mold having 3D contour (FIG. 4A) with a surface area of ˜250 mm². The cast was then positioned in the middle of 60 mm tissue culture while a 10:1 mixture of PDMS prepolymer and curing agent (Sylgard 184 silicone elastomer kit, Dow Corning, Midland, Mich.) was degassed and poured onto the cast. This dish was allowed to cure for 24 h in a laminar hood. The wound model was kept in the tissue culture dish so that cell culture media can be added after cell-hydrogel printing.

For the direct cell printing on the non-planar PDMS wound model, the desired printing patterns were obtained from the CAD file (Solidworks, Concord, Mass.) of the model, and its spatial dimension and shape were used to plan the 3D printing patterns in multiple layers (a sequence of the printed planar layer for the model is shown in FIG. 4C). The distance between the nozzle and the target substrate was maintained at 5 mm. Another optional mode of printing, although not used in this experiment, was to follow the contour of the non-linear surface while filling non-planar surface. Although the collagen precursor was dispensed onto the curved surface of the PDMS mold, the surface treated with nebulized NaHCO₃ cross-linked collagen, and retained the subsequent layers of printed morphology.

### Example 5

### On-Demand Non-planar Multi-Layer Printing and Culture of hFB and hKC for 3D Skin Wound Model

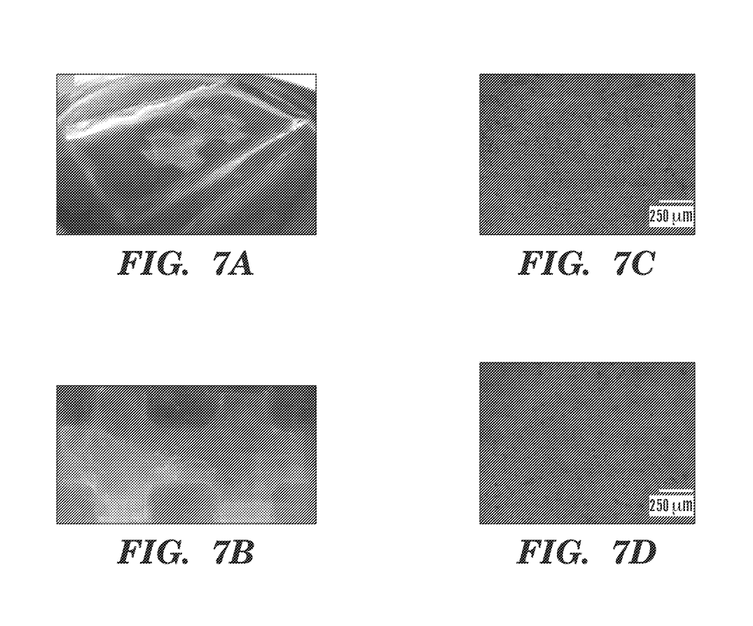

FIG. 7 shows the results obtained from multi-layered printing of hFB and hKC on non-planar PDMS surface mimicking a 3D skin wound model. hFB and hKC layers were embedded in the 2nd and the 8th layers of collagen scaffold from bottom, respectively. FIGS. 7A and 7B are the images of printed cell-collagen composite on the PDMS mold. The surface of cell-collagen was wrinkled due to the cell suspension printing over collagen layers and the cell attachments in collagen scaffold. The inter layer distance of hFB and hKC layers at the concave area was approximately 100 μm, however that of the convex area was reduced to approximately 60 μm. FIGS. 7C and 7D show bright field images of hKC and hFB layers located in a same field-of-view (pictured in Day 1). Both bright field images of hKC and hFB layers show varying depth of focus from upper left area to lower right area, which show the 3D contour of the PDMS mold surface.

### Example 5

### Construction of Multi-Layered Hydrogel Channels

For generating multi-layered hydrogel channel, collagen hydrogel precursor (Rat Tail, Type I, BD Bioscience, MA) was diluted 1:1 with PBS while maintaining a pH of 4.5. Undiluted Collagen hydrogel was too acidic (pH-3) to cultivate biological cells. For cross-linking material for collagen, 0.8 M NaHCO₃ solutions was used (prepared in distilled water, concentration 71.2 mg/mL; according to Gangatirkar et al., (Nat. Protoc. 2007, 2:178-186). Gelatin (Porcine skin Type A) was prepared as solution (7 weight %) as a sacrificial material, and heated to 40° C. and stored in a heated dispenser unit.

A schematic shown in FIG. 8 illustrates the method of constructing multi-layered (5-layer) hydrogel composites with patterned sacrificial gelatin channels. First, the surface of the Petri dish was thinly coated with nebulized aerosol of NaHCO₃ solution (less than 2 μm in diameter). The coating was crucial to bind the subsequently printed collagen precursors to the dish surface and initiate gelation. Then, an initial layer of collagen was printed on an area of 10 mm by 10 mm square. NaHCO₃ solution, again as an aerosol, was applied on the top surface to cross-link remainder of printed collagen bed. Coated NaHCO₃ also served as the binder and cross-linker for the subsequent collagen. In the next layer (layer #2), collagen was patterned while leaving the space for the gelatin channel. After the cross-linking collagen pattern, gelatin was printed on the groove (FIG. 8).

In FIG. 8, after planar layer (steps 1 and 4) and groove form (steps 2 and 5) of collagen hydrogel was printed and gelated (by cross-linking agent, sodium bicarbonate), sacrificial gelatin channel was printed into the collagen groove and gelated (by cooling under 20° C., 10˜20 min) (steps 3 and 6). One more planar collagen layer was printed and gelated to cover the 2nd gelatin channel (step 7). The constructed 3D collagen-gelatin hydrogel structure was kept in incubator (36.5° C., 20 min), and then the gelatin channels were selectively liquefied (step 8). By perfusing warm liquids such as phosphate buffered saline or cell culture media through the gelatin channels, the liquefied gelatin was completely removed and the multi-layered fluidic hydrogel was constructed (step 9).

Collagen layer (layer#3) was printed on top of the channel containing hydrogel layer to seal the channel space. The process repeated again to print the different shapes of collagen-gelatin channels (‘X’ shape in the FIG. 8) while the vertical stage was lowered to maintain the distance between the dispenser and target.

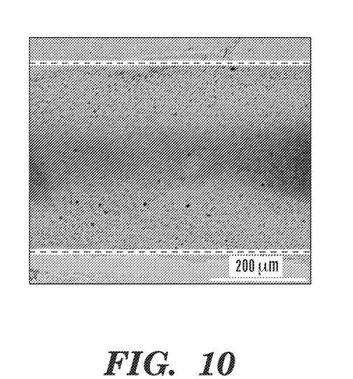

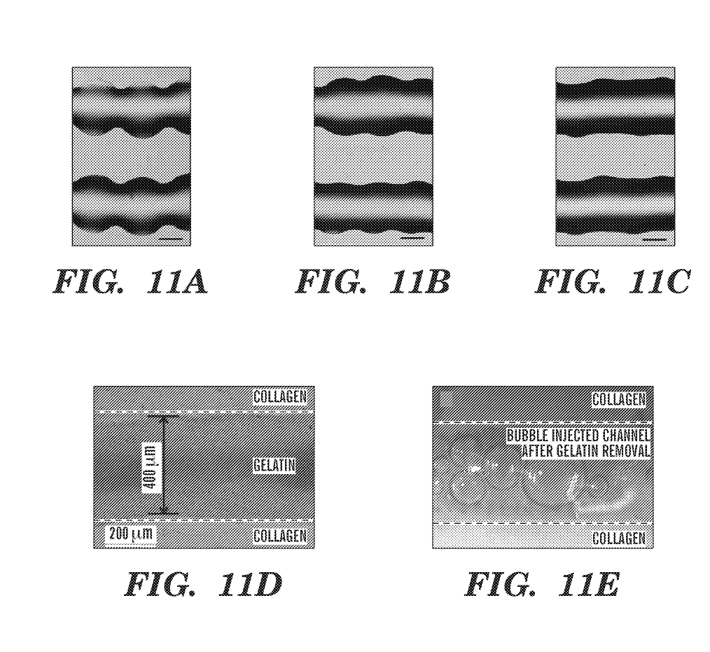

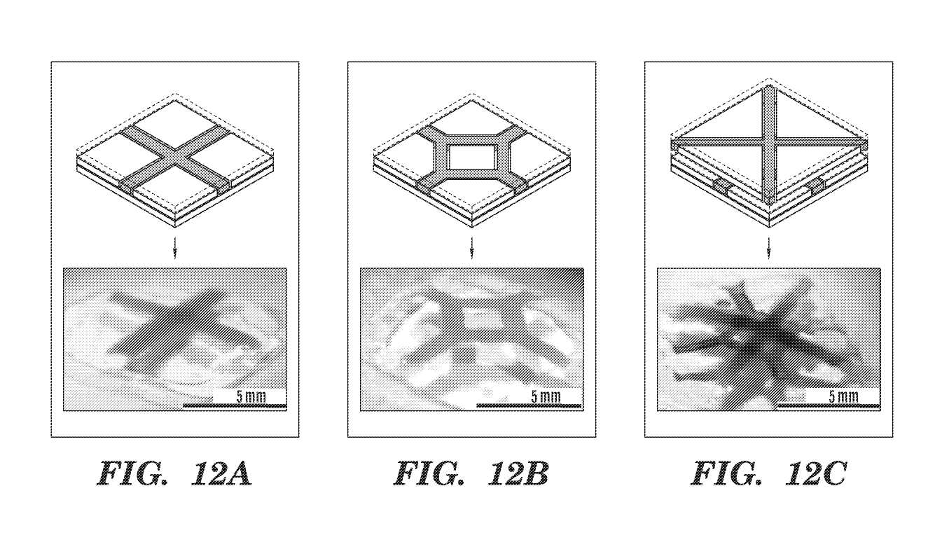

FIG. 10 shows a gelated gelatin channel in collagen groove. After the completion of the channel-containing hydrogel block (example shown in FIG. 12 consisted of 5 layers), the structure was subsequently heated to 40° C. (via TED in the target substrate) so that gelatin in the channel was carefully removed using syringe needle. The channel created by the space occupied by the gelatin was filled with distilled water containing colored microspheres (Bangs Laboratory) to visualize the channels.

In order to examine the potential utility of hydrogel channel for the application in tissue engineering, a separate set of tissue construct containing a single straight channel in the middle (3rd) layer was prepared, and subjected to the different hydrostatic pressure to examine its integrity. The one end of the channel was connected to the inlet of the channel and other end was closed using the cross-linked collagen plug. The pneumatic pressure was increased from 0 psi to 2 psi (104 mmHg) with the step of 0.2 psi and channel structure was examined for presence of any leak or the crack in the collagen gel.

Spatial resolution of 400 μm was used in dispensing collagen to construct the each 10×10 mm hydrogel layer. It took approximately 1 minute to generate each layer, including the time necessary to the cross-link the hydrogels after printing. Using the described robot-assisted 3D fabrication technique, various channel structure in the collagen gel was constructed, as shown in FIG. 12. Non-crossing channels were selectively visualized using water-insoluble colored micro-beads. Based on the examination of lumen pressure of the hydrogel channel, the 10 mm-length channel in 0.5× collagen structure resisted up to 104 mmHg (=2 psi), which can withstand the average blood pressure in artery (normally 80˜120 mmHg). In addition, a 12 collagen layers construct was printed without any structural collapses (height of structure was 800 μm).

In order to demonstrate the versatility of the method, 2-dimensional crossing channel pattern (FIG. 12B) and ‘rotary’-shaped channels (FIG. 12C) were also prepared using 3-layered printing. Straight channels that are not overlapping each other were also prepared using 5-layer hydrogel structure. The top layer was sealed with collagen layer using the same gelation procedure.

### Example 6

### Testing of Cell Viability in Perfused Hydrogel

The ability to perfuse the cells via the channel was also examined. A straight hydrogel channel was made in a multi-layered 3D construct of 10×10×2 mm³ with fibroblasts embedded across the volume by printing the cells in each layers, starting with the 2nd layer collagen. In order to ensure that the bottom middle layer of cells, without the channel, is away from the passive diffusion across the hydrogel, a total of 17 layers of the collagen gels were constructed to form a hydrogel, resulting in maximum thickness of ˜2mm. A tissue construct without any channel, as a control condition, was also prepared at the same time. Subsequently, the channel was connected to the syringe pump (NE-1000, New Era Pump Systems, Wantagh, N.Y.) and perfused with fibroblast media (Sciencell Laboratory, CA) at a rate of 1.5 μL/min. There after the construct was cultured in normal culture condition (5% CO₂ and 37° C.) for 36 hours, and cell viability was examined using LIVE/DEAD Viability/Cytoxicity Kit (L-3224, Invitrogenl Calcein-AM and ethidium homodimer-1) under the fluorescent microscope (Model #; Nikon, Japan).

In another modified method for studying cell viability, a 60 mm tissue culture dish with a hole (for infusion) was prepared whereby the bottom of the dish was penetrated by a 30½-gauge syringe needle. The needle outlet (made blunt by cutting and polishing the end) was connected to a loaded syringe through a Tygon tube (see the schemes in FIG. 14 middle, right). The assembled tissue culture dish was sterilized by UV radiation for >30 min, and then a 17 layered collagen hydrogel block containing a single straight channel were printed on 10×10 mm² square area. The straight line of gelatin (for channel creation, ˜400 μm in width and 110 μm in height) was printed in the midline of 2nd collagen layer from bottom and aligned to intersect the infusion inlet (see schemes of FIG. 14B and 14D). During the construction, collagen was printed with a resolution of 400 μm at pressure of 2 psi and a valve opening time of 600 μs. Gelatin was printed on the collagen groove twice at a printing resolution of 150 μm under an operating pressure of 6.7 psi and a valve opening time of 750 μs. hFB (1×10⁶ cells/mL) were embedded in the scaffold by printing the cells in each of the two layers, starting with the 2nd collagen layer from bottom. Thick FB-laden collagen composites were necessary to examine the effects of perfused channel on the cells located beyond passive diffusion limitation (on the order of 1000 μm according to the (Ling et al. 2007, Lab Chip 7:756-62).

Once printing was completed, the collagen-gelatin structure was kept in an incubator to liquefy and remove the gelatin. Then, warm FB culture media was perfused into the fluidic channel inside collagen scaffold at a rate of 4.0 μl/min using a syringe pump (NE-1000, New Era Pump Systems, Wantagh, N.Y.). Both ends of the channels were not plugged, which allowed for free flow of the media through the channel. As a comparison with the perfusable collagen scaffold, an identical FB-laden collagen scaffold, without the channel, was prepared. These two FB-laden collagen scaffolds were submerged in 5 mL of FB culture media, and cultured in 5% CO₂ at 37° C. After 36 hours of culture, a live/dead viability/cytotoxicity assay (L-3224, Invitrogen Calcein-AM and ethidium homodimer-1) was conducted.

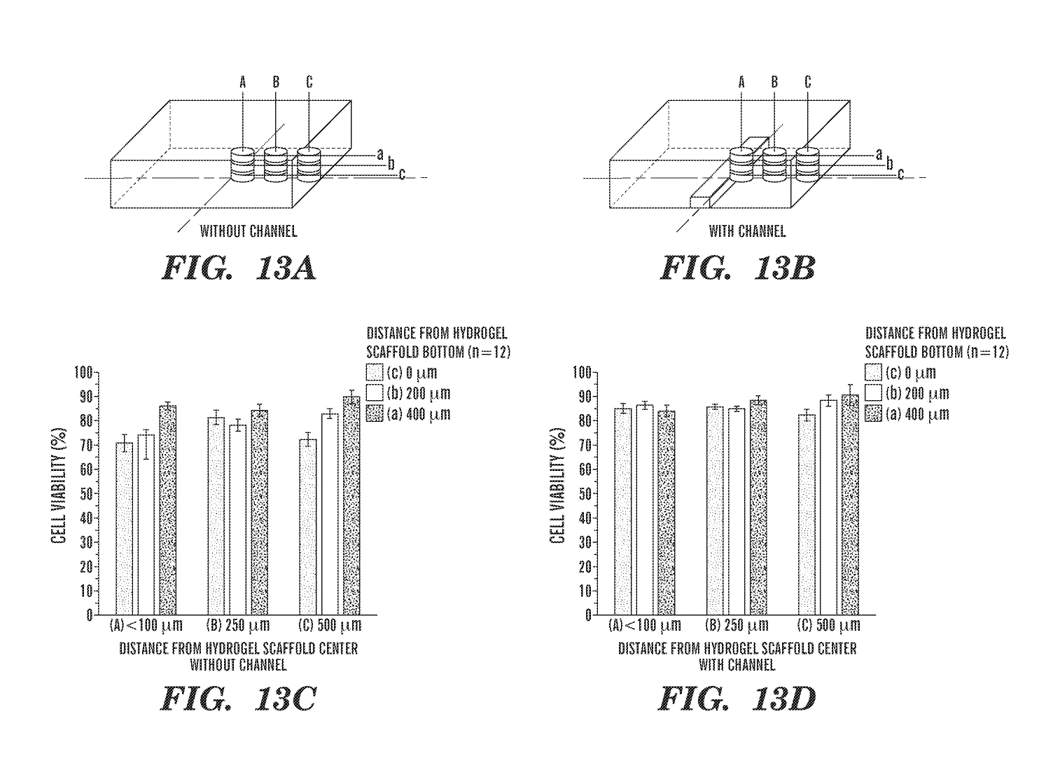

The hydrogel block with embedded fibroblasts had thickness approximately 1500 μm. The cells were well-attached and uniformly spread in the dispensed 3D collagen structure. FIG. 13 shows the viability testing results obtained from the set of areas-of-interest consisting of 500×500 μm² radial to the channel structures across the three different surface depths (at the surface, on the level of channel and in the middle layer). The fibroblasts located in the top layers of the 3D hydrogel showed the high viability great than 80%. The level of similar viability was observed form the middle layers due to the passive perfusion up to 1 mm in depth. However, the viability of fibroblasts located in the bottom layers in the control hydrogel, without the channel, was greatly reduced (down to approximately 70%), while the hydrogel with channel showed the quite uniform distribution of the viability across all gel structure.

Similar results were obtained using the modified method for studying cell viability. The printed collagen that contained a straight channel resisted over 103.4 mmHg (=2 psi=13.79 kPa) of hydrostatic pressure without any leaks and cracks of the collagen scaffold. The collagen scaffolds (consist of 17 layers of cell/hydrogel) with embedded FB had a thickness of approximately 1450 μm as measured by adjusting the focal distance of the microscope between top and bottom of the scaffolds.

The printed FB cells were initially well-attached and uniformly spread in both collagen scaffolds and no contamination was observed after 36 hours culture. Viabilities of FB in collagen scaffolds after 36 hours culture with and without perfusion are shown in bottom of FIG. 14. The regions-of-interest where the viability of FB was measured (middle of FIG. 14) were positioned on cross-sectional area at M-M′ (FIGS. 14C and 14D) and radial from the perfusion channel. In region-by-region comparisons between the two scaffolds with and without the perfusion, the FB located in the top layer (FIG. 14 layer (a)) of the both collagen scaffolds showed high cell viability (greater than 80%). However, the viability of FB in layers (b) and (c) in the middle of the non-perfused collagen block was reduced significantly compared to the ones measured from the collagen block perfused using the channel. It was also observed that the collagen block with the channel showed a uniform distribution of high cell viability (FIG. 14).

Hence, it was demonstrated that the fibroblast embedded in the thick hydrogel block showed high cell viability to the depths around the channels. This indicates that adequate perfusion to the cells were provided by the syringe pump via the channel. The example demonstrated that the 3D bioprinting alone can construct both the artificial tissue and hydrogel channel embedded within.

### Example 7

### Quantification of Droplet Volume and Channel Width

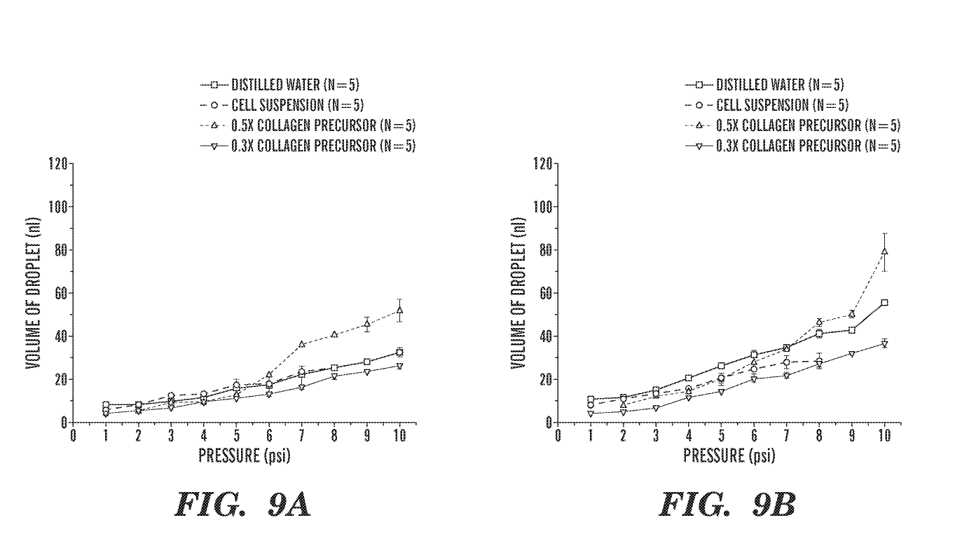

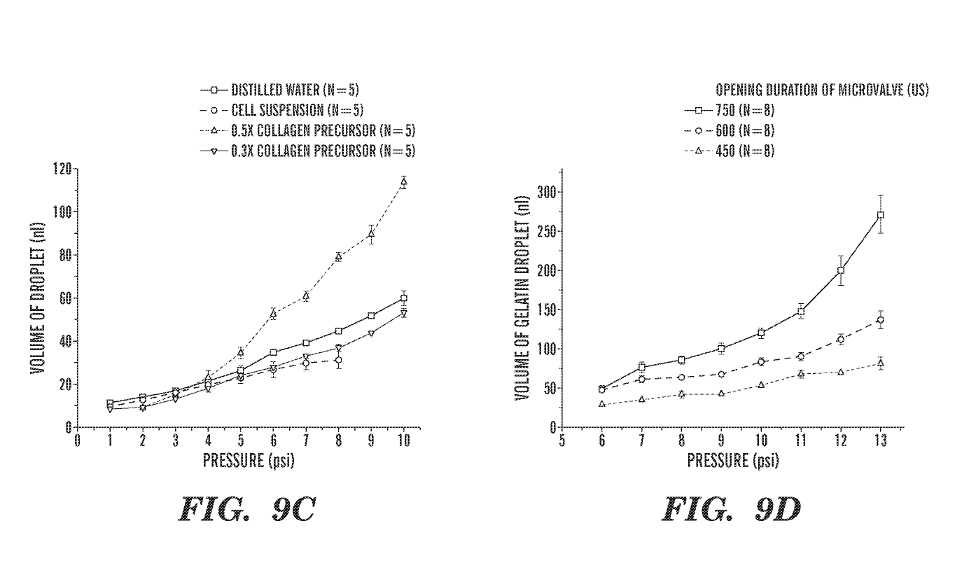

The relationship between dispensed droplet volume of 7% (w/v) gelatin (at 40° C.) and valve opening time and applied pneumatic pressure were studied. FIGS. 9A-9C show the droplet volume of distilled water (DW), fibroblast-containing media, uncross-linked collagen solution with different dilution factor (1:1 and 1:2) for different valve opening time and applied pneumatic pressure. The volume of the droplet, regardless of the type of used material, was dispensed in the range of 5 nL-30 nL. As anticipated, longer valve opening time and increasing pressure resulted in dispensing of larger droplet volume. Collagen precursor in 1:2 dilution factors was less viscous than that of 1:1 dilution, and smaller droplet at given pressure and valve opening time was possible.

Gelatin (at 40° C.), being more viscous compared to the other tested material, was dispensed at higher pressure level, around 6 psi. The higher pressure, compared to dispensing the cell-containing media and DW, was needed to overcome the surface tension of the nozzle. Pneumatic pressure less than 6 psi often resulted in deviation from the straight dispensing path or formation ‘satellite’ droplets that affect the printing accuracy. Minimum droplet volume was estimated to be 25 nL; at valve opening of 450 μs (FIG. 9D). Increase in pressure and valve opening duration increased to volume as much as few hundreds nanoliter.

The printing resolution for a given droplet size can influence the width and homogeneity of the printed gelatin pattern since each droplet will conglomerate after landing on the substrate surface. FIGS. 11A-C demonstrates the example of such influence. When we examined the shape of printed gelatin line by changing the printing resolution (300 μm, 400 μm, and 500 μm) at 4 psi, larger printing distances (>500 μm in a given dispensing condition) generated uneven line shape (FIG. 11A).

FIGS. 11D and 11E show the printed straight gelatin line and the gelatin-removed fluidic channel in multi-layered collagen blocks, respectively. At the dispensing condition of the gelatin (pressure: 6 psi; valve opening duration: 450 μs; printing resolution: 700 μm), a channel width of approximately 400 μm was achieved. Since the patterns were created based on the sequential dispensing of the gelatin droplets, the line width was slightly inhomogeneous (typically less than 20 μm). After removing the gelatin from the collagen scaffold, air bubbles were intentionally injected using a 30½-gauge needle into the formed channel to visually confirm the channel construction (FIG. 11E). colored microbeads was loaded inside the channel for the visualization to measure the channel height (−110 μm through the adjustment of the focal depth of a microscope).

The use of electromechanical valves for the dispensing hydrogel was effective for constructing proposed channel structure. The low pneumatic pressure (<5 psi) and passive gating of the fluid path was also conducive to have high cell viability if the cells need to be embedded simultaneously. Since the sacrificial gelatin channel was created based on the sequential dispensing of the droplets, there was degree of non-homogeneity of channel width. However, use of the different dispensers with capability to dispense picoliter-nanoliter volume droplet can reduce the variability of channel width with potentially much smaller channel width. The required increased in spatial resolution and printing speed can be addressed by multiple, closely-arranged array of dispensers.

In conclusion, a new method to construct chemically-cross-linkable 3D hydrogel channels using on-demand freeform fabrication technique is demonstrated here. The coating of the each layers with nebulized cross-linking agents were crucial for the multi-layered construction of the hydrogel. The process can be repeated to stack multiple layers of the hydrogels with complicated printing patterns that encapsulate cells and other bioactive agents. The described CAD-tissue printer is capable of successfully storing and dispensing both chemically cross-linking and thermal cross-linking hydrogels. The addition of the temperature regulated dispenser to one of the electromechanical dispenser allowed the dispensing of thermo-sensitive hydrogel in a liquid form. A construct of up to 17 layers of collagen-based hydrogel with the height of 2 mm in height was constructed without presence of structural crack or the collapse.

### Example 8

### 3-D Bioprinting of Rat Embryonic Neural Cells

The construction of single/multilayered cell-hydrogel composites were as described herein. During this process, the generation of ultrafine mists with droplets less than 2 mm in diameter (when landed on the culture dish surface, as measured by microscope) was crucial to crosslink the dispensed collagen precursors (in the order of 200-300 μm in diameter) without distorting the printing morphology because of the surface tension of dispensed droplets. The cell suspension was then printed on the partially-crosslinked hydrogel layer to lodge the cells inside the collagen. Each collagen layer was printed to occupy a 10×10 mm² area using the interdispensing distance (i.e. printing resolution) of 600 μm.

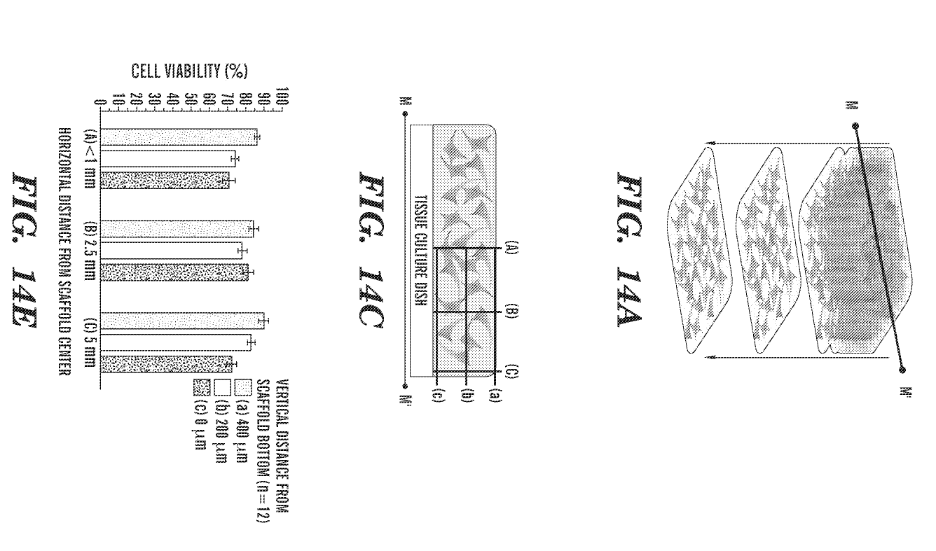

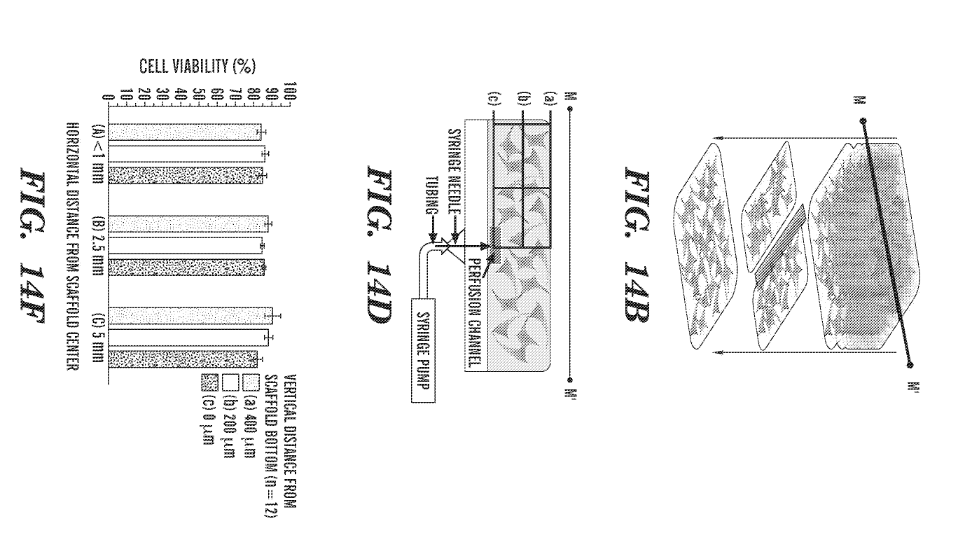

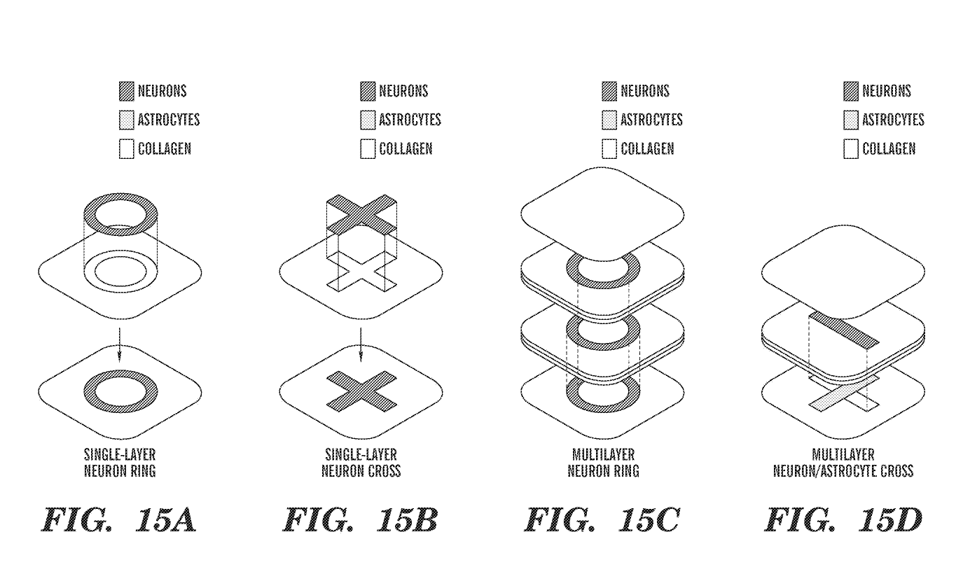

Testing of neural cell printing resolutions was conducted. Before the multilayered neural cell-hydrogel printing, the relationship between printing resolution and the growth tendency of cells was investigated. Six different printing resolutions (150-400 μm in 50 μm step) were examined for printing neurons in a single layer of collagen (measuring 5×5 mm²; n=3), whereas three different printing resolutions (200, 400, and 600 μm) were examined for astrocytes. After printing, the cells were monitored using bright field microscopy (for astrocytes) or green fluorescent live staining (Calcein AM; for visualization of neurites through the semitransparent collagen scaffold). Based on the examination of growth pattern (FIG. 16), a resolution of 150 μm for neurons and 300 μm for astrocytes were selected for subsequent printing experiments. Printing and culture of neural cells in single-layered and multilayered hydrogel scaffolds Neurons were printed and cultured in a ‘ring’ pattern (3 mm diameter; FIG. 15A) and a ‘cross’ pattern (two 6 mm long orthogonal lines; FIG. 15B). To generate multilayered cell-hydrogel composites, a total of eight layers of collagen were printed (FIG. 15C). Rings of neurons were separated by the two layers of collagen, which were sandwiched between printed rings of neurons. A multilayered ‘cross’ pattern vas also printed consisting of astrocytes and neurons (FIG. 15D). To test the feasibility of printing two types of cells into the same area for coculture, both astrocytes and neurons were printed as a single layer in the middle of the collagen scaffold (3×3 mm²). Neurons were printed at slightly lower resolution (200 μm) to account for the added astrocytes.

After printing, the neural cell-collagen composites were cultured at 37° C. and 5% CO₂ in Neurobasal media with 2% B27 supplement, 0.5 mM glutamine, and 25 μM glutamate. Half of the media was replenished with fresh media (without glutamate) every 3 or 4 days, and the cells were cultured for a maximum of 15 days. The printed cell-collagen composites were immunostained using microtubule-associated proteins 2 (dilution factor 1:250; Santa Cruz Biotechnology, Inc.) for labeling neurons and Glial fibrillary acidic protein (1:200; Santa Cruz Biotechnology, Inc) for labeling astrocytes according to the vendor-suggested protocol at the Cell Signal World Wide Web site. Subsequently, Texas red fluorescence-labeled secondary antibody (1:100; donkey anti-rabbit, Jackson Laboratories, Inc.) was applied for labeling the neurons. Fluorescein isothiocyanate fluorescence-labeled secondary antibody (1:100; goat anti-mouse, Jackson Laboratories, Inc.) was used for the astrocytes. To increase the penetration of antibodies into the scaffold, the sample was placed on a rocker (frequency 30 rpm) during all procedures without using any cover slide. To visualize the neurite outgrowth in a thick (in the order for several hundred micrometers) and semitransparent hydrogel, we adopted the visualization method proposed by O'Connor et al. 2001, (Neurosci. Lett., 304:189-19) and Othon et al. 2008, (Biomed. Mater., 3:034101) whereby the ‘stacks’ of multisliced confocal images (Obtained from LSM 510 confocal with two photon, Carl Zeiss) were digitally projected along the vertical direction (so called ‘Z-stacking’ technique) to capture the 3D representation of the cell morphology.

Droplet size and viability assay of printed neural cells were investigated. When measured through high-speed camera (Pixelink), the droplet volume of dispensed cell suspension and collagen precursor was approximately 11 and 8 nl, respectively. The number of cells contained in each droplet was 217.8±21.6 cells for neurons (n=12) and 49.8±4 cells for astrocytes (n=4). The viability of neurons (control) was 75.2±2.3% (n=32) while printed neurons showed a viability of 78.6±0.6% (n=34). The viability of astrocytes (control) was 78.7 ±5.3% (n=12) while printed astrocytes showed a viability of 78.1±10.0% (n=12). There was no significant difference in the viability of printed neural cells compared with the control group (P>0.05; t-test, two-tailed), suggesting that the cell printing did not affect cell viability. Investigation of printing spatial resolutions Day 15 culture images of FIG. 16A and 16B showed a difference in density of cultured neurons at printing resolutions of 150 and 250 mm interdispensing distance.

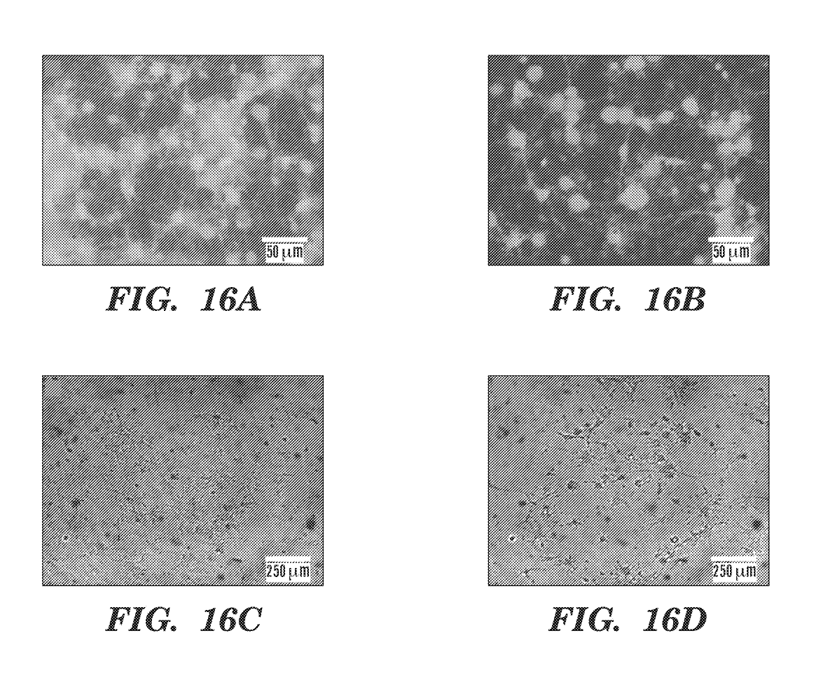

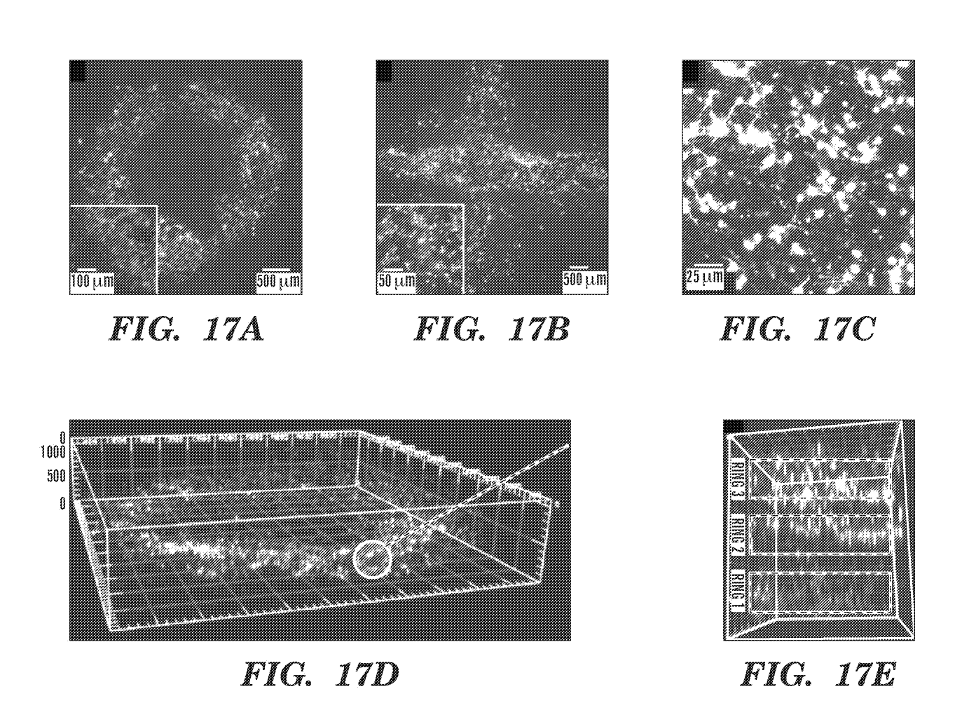

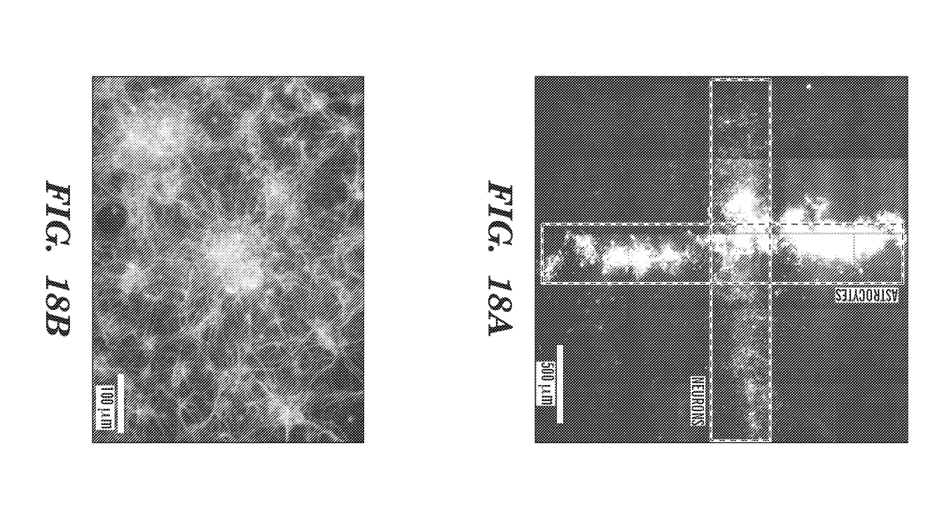

The neurons printed at 250 μm resolution (FIG. 16B) were more sparsely distributed compared with the neurons printed at 150 μm resolution (FIG. 16A), which showed the elevated cell density and neural connectivity through neurite outgrowth. The neurons printed at a low printing resolution did not display visible neurite outgrowth within 10 days. The astrocytes printed at a resolution of 600 μm showed a slow growth rate (FIG. 16D) compared with the ones printed at a resolution of 200 μm, which reached excessive confluency. However, printing resolution of 400 mm lead to a sufficient growth rate and morphologies of astrocytes (FIG. 16C). Culture of printed neural cells in single-layered and multilayered collagen scaffold Mosaic fluorescent images of the printed neural cells are shown in FIG. 17. The ring and cross pattern of live neurons in a collagen layer are shown in FIG. 17A and 17B, respectively. The multilayer pattern of three neuron rings was shown in a 3D-rendered microtubule-associated proteins 2 immunostaining image (FIG. 17D). As evident from the reconstructed side view (inset FIG. 17E), three distinct layers of rings of neurons were distinguished. Patterned neurons showed neurite outgrowth and neural connectivity in three dimensions, based on the projected multistack confocal image (FIG. 17C).

The immunostaining results obtained from the neurons and astrocytes that were printed on a single-layer collagen scaffold were shown in FIG. 18. As anticipated, the star-like morphology of astrocytes, which is typically observed on planar substrates, was slightly distorted in the volumetric collagen gel (Gottfried C, et al., 2003, Neuroscience, 121:553-562). FIG. 18A shows a multilayered pattern of neurons and astrocytes stained with 4′-6-Diamidino2-phenylindole staining to visualize the macroscopic location of the printed cells through a thick hydrogel scaffold. The clusters of both neurons and astrocytes were visible in the middle as well as in the lower left corner of FIG. 18B.

### Example 9

### 3-D Printing of Collagen and VEGF-Releasing Fibrin Gel Scaffold for Neural Stem Cell (NSC) Culture

To support the growth and differentiation of the NSCs in culture condition or at an implanted site of body tissue, introduction of appropriate epi-cellular environments, such as mechanical support, growth factors, and surface modification for cell-attachment and proliferation, is needed. Therefore, cells are typically introduced to the target region by either being mixed with or being seeded on a biodegradable ‘scaffold’. Here the inventors apply the freeform cell printing for cell replacement therapy which aims to introduce artificially constructed biological tissue/cells to the site of neural tissue damage with an ultimate goal of replacing damaged or injured neural tissues while potentially addressing the wide ranges of neurological diseases involving central nervous system.

To determine the effects of VEGF, a combined 3D freeform collagen scaffold and VEGF-containing fibrin gel was constructed. The scaffold was printed on a 60 mm tissue culture dish. The channel assignments and parameter settings of the materials are as follows; collagen scaffold precursor with a pressure (p) of 2.2 psi and a valve opening time of (Δ) 500 μs, fibrinogen printing solution containing VEGF at p=5.0 psi and Δ=500 μs, thrombin printing solution containing VEGF at p=3.0 psi and Δ=500 μs, C17.2 cell suspension at p=1.1 psi and Δ=500 μs.

VEGF delivering samples were printed by first printing 10 μL fibrinogen printing solution containing VEGF in a circle pattern (5 mm diameter). On the same position, 10 μL thrombin printing solution with VEGF (5 μL thrombin solution plus 5 μL VEGF solution) was printed immediately over the area to create the fibrin gel. The bottom layer of collagen was printed directly onto it in a square pattern (14×14 mm²). C17.2 cells were printed beside the fibrin gel border in a rectangular pattern (3×2 mm²) and the top layer of collagen was printed in a square pattern (14×14 mm²). The final concentrations of the printed fibrin gel were as follows; 31.4 mg/mL for fibrinogen, 66 U/mL for aprotinin, 66.6 NIH U/mL for thrombin, 2.38 μg/μL for heparin, 5.9 mg/mL for CaCl₂, 50 ng/μL for VEGF.

For the control conditions, cell-hydrogel composites with fibrin gel that did not contain VEGF were also constructed. Here, the same procedure was followed, except the volume of VEGF solution used was replaced with the equivalent volume of the fibrin gel precursor solution or thrombin solution. As the second control condition, a cell-hydrogel composite using collagen only was prepared; however, 10 μL of the VEGF solution was patterned in the same location with respect to the printed NSCs. The completed scaffolds were incubated at 37° C. for 15 minutes and 100-150 μL of serum-free media was carefully placed on top of each of the scaffolds after incubation. The tissue culture dish containing the scaffold was then placed in the 100 mm tissue culture dish filled with 20-25 mL of distilled water to prevent potential drying.

The morphology of C17.2 cells in culture has been well characterized (Niles et al., 2004, BMC Neurosci. 5:41-41). Undifferentiated C17.2 cells have a flat and rounded appearance while differentiated C17.2 cells have an elongated shape with an extension of neurite-like processes (Niles et al., 2004, supra). The morphology of C17.2 cells was observed using bright-field microscope at 0, 1, 2, and 3 days after being printed onto the collagen scaffold. The images were analyzed for the stability of the hydrogel as well as for signs of morphology changes and migration of cells due to the VEGF-releasing fibrin gel embedded in the collagen scaffold. To monitor the same region-of-interest in the scaffold, small dots were marked on the bottom of the tissue culture dish. Montage images were made from the pictures of the area. Using ImageJ (National Institute of Health, Washington D.C.), the movements of C17.2 cells in the scaffold were evaluated by measuring movement path towards the border of fibrin gel containing VEGF between each observation time point.

The mean number of C17.2 cells contained in each dispensed droplet at 1×10⁶ cells/mL of cell suspension was 56±9 cells/droplet, as measured using bright field microscopy (n=10). The viability of manually plated C17.2 cells was 91.68±1.84% and while printed cells showed a viability of 93.23±3.77%. There was no significant difference in viability of printed cells compared to manually plated cells (p>0.05; t-test two tailed; n=5) suggesting that the disclosed cell printing technique did not affect the cell viability.

The collagen scaffolds at various concentrations resulted in different proliferation patterns of cells, although all scaffolds were printed using the same resolutions for cell and collagen printing. The 1.74 mg/mL collagen scaffold showed cells proliferating most densely compared to ones in 1.16 mg/mL or 0.87 mg/mL collagen scaffolds (data not shown). The viability of C17.2 cells within each concentration of collagen was as follows: 96.72±3.58% of 1.74 mg/mL, 97.06±1.46% of 1.16 mg/mL, and 98.05±0.37% of 0.87 mg/mL at day 3. There was no significant difference in viability at each concentration of collagen (p>0.05; one-way ANOVA; n=5). The cells were viable up to 11 days (data not shown).

Effects of combined collagen scaffold and VEGF containing fibrin gel on C17.2 cells

The printed cell-hydrogel composites maintained structural integrity up to 7 days after printing. After 4 days, the viability of C17.2 cells in the collagen scaffold under media containing the serum was 92.89±2.32% (n=5). Cells were observed to proliferate and had an elongated shape with an extension of neurite-like processes that were similar to those seen in the scaffolds submerged in media containing serum. In contrast, the cells in collagen scaffolds in the serum-free media did not proliferate (data not shown). This result showed that serum is an important component in the viability, proliferation, and differentiation of NSCs within the scaffold and was largely expected as previous literature (Niles et al., 2004, BMC Neurosci 5, 41-41).

In the collagen scaffold containing fibrin gel and VEGF, the proliferation and change of morphology of C17.2 cells were observed during the 3 day monitoring period. After printing, C17.2 cells had a small, round appearance. Following day 1, the cells started to grow with changes in its shape (i.e. flattened). On day 2, some differentiating C17.2 cells were observed, as indicated by an elongated shape with an extension of neurite-like processes and on day 3, the differentiating cells were more frequently observed.

C17.2 cells observed to change morphology and to proliferate were within about 1,000 μm distance from the border of VEGF containing fibrin gel. The longer the cells were cultured, the further the border between the cells that are displaying these changes and the ones that have not changed progressed as follows: 195 μm for 3 hours, 483 μm for 1 day, 583 μm for 2 days, and 698 μm for 3 days. There also were several groups of cells moving towards each other and making clusters of cells or changing their morphology. The cells in the scaffold without the VEGF did not show any signs of cell differentiation across the observed time points. The cells printed with VEGF without the fibrin gel did not differentiate over time and began to shrink (data not shown).

The signs of cell migration toward the VEGF-containing fibrin gel were observed as well. The migrating cells were located mainly about 500-1,000 μm from the fibrin gel border. After one day, the changes in cell morphology and the proliferation of cells made cell tracking difficult in some regions. The mean moving distance toward fibrin gel during 3 days were: 33.33±18.16 μm during the first day (18 hours), 34.02±45.18 μm on the second day (24 hours), 33.12±46.52 μm on the third day (24 hours), with a total migration distance of 102.39±76.09 μm .

The references cited herein and throughout the specification and examples are herein incorporated by reference in their entirety.

1. A method of making a three dimensional multi-layered hydrogel construct, the method comprising the steps of:
a. applying a first nebulized layer of cross-linking material on a substrate;
b. depositing at least one layer of hydrogel precursor on top of the first nebulized layer of cross-linking material, wherein the hydrogel precursor cross-links upon contact with the nebulized layer of cross-linking material to form a partially cross-linked gel;
c. applying a second nebulized layer of cross-linking material on top of the partially cross-linked gel of step (b), thereby promoting completing cross-linking of the layer of hydrogel of step (b); and
d. repeating alternating step b followed by step (c).

2. The method of claim 1, wherein the hydrogel layer is deposited via drop by drop on-demand printing or continuous extrusion of the precursors.

3. The method of claim 1, wherein the nebulized cross-linking material comprises 1-100 micrometer sized droplets.

4. The method of claim 1, wherein step (d) is repeated 1-20 times.

5. The method of claim 1, wherein step (d) is repeated at least 5 times.

6. The method of claim 1, wherein step (d) is repeated at least 10 times.

7. The method of claim 1, wherein step (d) is repeated at least 15 times.

8. The method of claim 1, wherein the multi-layered three dimensional construct comprises more than one type of hydrogel.

9. The method of claim 1, wherein the hydrogel precursor is selected from a group consisting of collagen, gelatin, fibrinogen, chitosan, hyaluronan acid, alginate, poly-ethylene glycol, lactic acid, and N-isopropyl acrylamide.

10. The method of claim 1, further comprising depositing living cells on the layer of hydrogel precursor after step (b) but prior to step (c).

11. The method of claim 10, wherein more than one cell type is deposited in the multi-layered three dimensional construct.

12. The method of claim 11, wherein the cell types are selected from a group consisting of stems cells, pancreatic progenitor cells, neuronal cells, vascular endothelial cells, hair cells, mesenchymal cells, and smooth muscle cells.

13. The method of claim 2, wherein the substrate is flat.

14. The method of claim 2, wherein the substrate is contoured.

15. The method of claim 2, wherein the substrate is biological.

16. The method of claim 2, wherein the substrate is non-biological.

17. The method of claim 1 wherein the three dimensional multi-layered hydrogel construct further comprise of channels.

18. A three dimensional multi-layered hydrogel construct comprising at least 10 layers of hydrogel material, at least one type of cells, wherein the cells are deposited on different layers of hydrogel material, and at least one type of hydrogel material.

19. The three dimensional multi-layered hydrogel construct of claim 18 wherein the cells types are fibroblast and keratinocytes.

20. The three dimensional multi-layered hydrogel construct of claim 19 further comprising hair follicular stem cells.

21. The three dimensional multi-layered hydrogel construct of claim 18 wherein the cells types are vascular endothelial progenitor cells and smooth muscle progenitor cells.

22. The three dimensional multi-layered hydrogel construct of claim 18, wherein the cells types are pancreatic endothelial progenitor cells and mesenchymal cells.

23. The three dimensional multi-layered hydrogel construct of claim 18, wherein the cells types are neurons and astrocytes.

24. The three dimensional multi-layered hydrogel construct of claim 18, wherein the cells types are neural stem cells and astrocytes.

25. The three dimensional multi-layered hydrogel constructs of claim 18, further comprising bioactive agents.

26. The three dimensional multi-layered hydrogel construct of claim 18, wherein the cells are deposited on different layers of hydrogel material.
